(19) United States
(12) Patent Application Publication
(10) Pub. No.: US 2012/0045827 A1
(43) Pub. Date: Feb. 23, 2012

(54) SEPARATION OF ANTIGEN-SPECIFIC MEMORY B CELLS WITH A CONJUGATED BIOPOLYMER SURFACE

(75) Inventor: Darryl Sampey, Frederick, MD (US)

(73) Assignee: BIOFACTURA, INC., Rockville, MD (US)

(21) Appl. No.: 13/255,418

(22) PCT Filed: Mar. 9, 2009
(86) PCT No.: PCT/US2010/026624

§ 371 Date: Nov. 1, 2011

Related U.S. Application Data
(60) Provisional application No. 61/158,649, filed on Mar. 9, 2009.

(51) Int. Cl. C12N 5/078; C12M 1/00

(52) U.S. Cl. 435/325; 435/283.1

(57) ABSTRACT

The present invention provides materials and methods for the isolation of immune cells. In one embodiment, the present invention provides a device for the isolation and separation of antigen-specific memory B cells. Immune cells will be separated by flowing the cells along a ligand-conjugated biopolymer surface. The ligand may be distributed in a concentration gradient along the z-axis of the biopolymer surface. Cells that display receptors specific for the ligands on the biopolymer will interact with the ligands and roll along the surface in the fashion of leukocyte rolling. Additive adhesive interactions will cause differential cell separation and eventual immobilization.

### CROSS-REFERENCE TO RELATED APPLICATIONS

This application claims priority to U.S. Provisional Application for Patent No. 61/158,649, filed Mar. 9, 2009, the entire contents of which are specifically incorporated herein by reference.

### BACKGROUND

The potential use of viruses (e.g., poxviruses, filoviruses) as bioweapons is a growing concern, especially with regard to human diseases for which effective countermeasures are presently unavailable. Administration of therapeutic antibodies represents a relevant strategy for treatment and/or prophylaxis of individuals exposed to or infected by viral disease agents of significance to the military and anti-bioterrorism efforts. Currently, vaccinia virus (VACV) immune globulin (VIG), a human blood-derived polyclonal Ig product, is the “first line” therapy for treatment of disseminated VACV infection stemming from adverse smallpox vaccination events. In addition, a cross-reactive mAb formulation could serve as a therapeutic or prophylactic for infections caused by other pathogenic orthopoxviruses such as monkeypox virus and variola virus, the causative agent of smallpox, thereby mitigating the threat of bioterrorism with such viral agents.

Of the approximately 200 genes contained in the VACV genome, only a few encode proteins that are known to induce a neutralizing antibody response, including H3, A17, A27, D8, L1, and B5¹. A seventh gene, A33R, encodes a protein that elicits a non-neutralizing antibody response but is, nevertheless, protective. Based historic and preliminary data, it is likely that combinations of VACV antigen-specific mAbs that exhibit protective efficacy in animal models will be effective for treatment of disease in humans. One method used to identify Fabs to A33R, B5R, and L1R is the pCOMB3 phage display system (Scripps Institute). This method can be problematic, for example, due to low library size/diversity, inefficient cloning, and stability problems associated with the first-generation pCOMB3 system.

With the approval of Humira in 2003, the industry has seen a full shift of new antibody therapeutics from chimeric to humanized to fully-human sequences. The objective in the industry is to manufacture a drug that is identical to that which is produced in the human body. Currently available methods suffer from various problems, for example, the high cost of discovering and manufacturing antibody-based drugs and difficulties with speed, stability, fidelity, regulatory compliance, and product expression.

Immunotherapeutics such as monoclonal antibodies have been proven to be lifesaving medical products and key reagents for diagnostic and research tools. There remains a need in the art for improved methods and devices for the identification and production of fully humanized monoclonal antibodies having a desired specificity. The present invention is exceptionally suited to the task, as it will harness human B cells. These cells are uniquely designed to express and secrete antibodies. The genetic, biochemical, and cellular organelle composition of cell lines derived from human B cells will accurately and faithfully produce the most human therapeutic molecule possible. The present invention also provides a device for the isolation of antigen-specific memory B cells. This device may be used for the discovery and manufacture of new generation immunotherapeutics to meet critical needs in areas such as infectious disease, oncology, public health, and biodefense.

### BRIEF SUMMARY OF THE INVENTION

Highly potent human therapeutic monoclonal antibodies can be identified and manufactured with isolated antigen-specific clones derived from the circulating memory B cell compartment of a human donor using differential adhesion mediated cell separation on an antigen conjugated biopolymer surface. Foreign (viral) antigen-specific memory B cells can be isolated from vaccinated donor human peripheral blood mononuclear cells (PBMCs) using indirect affinity magnetic bead methods.

In one embodiment, the present invention provides a device for the separation of immune cells by interactions of receptors of said immune cells with ligands. Typically, the ligands are immobilized on a functionalized surface of the device and the device is adapted to flow the immune cells across the functionalized surface. Any type of ligand may be used, for example, ligands bound by receptors present on the immune cells. Suitable examples include, but are not limited to, antigens for which the immune cells are specific. In some embodiments, devices of the invention may be flow cells and/or microfluidics device.

Devices of the invention may be used to isolate any type of immune cell, for example, neutrophils, eosinophils, basophils, lymphocytes, and/or monocytes. In some embodiments, cells to be isolated using the device of the invention may be lymphocytes, for example, B cells, T-cells, and/or natural killer cells. In some embodiments, cell to be isolated may be B cells.

Any receptor expressed on the surface of an immune cell may be used to isolate the cell by attaching one or more ligands bound by the receptor on the functionalized surface. Suitable receptors include B cell receptors for which suitable ligands include, but are not limited to antigens with B cell epitopes. In another embodiment, the immune cell receptors may be T cell receptors for which suitable ligands include, but are not limited to, antigens with T cell epitopes.

Any suitable method known in the art may be used to functionalize the surface of the device upon which immune cells are to be isolated. In some embodiments, the surface may be functionalized by depositing a polymer on the surface. One or more ligands may be conjugated to the polymer before or after deposition.

The present invention provides methods of isolating immune cells by contacting a solution comprising the immune cells with a device of the invention. In some embodiments, methods of the invention may comprise arresting (i.e., immobilizing) immune cells the functionalized surface. Such immune cells may be caused to proliferate by induction of activation. Such activation may result in the formation of colonies of immune cells. In some methods, viable activated proliferating immune cell colonies are recovered from the device. Colonies may be recovered using any method known in the art, for example, colonies may be recovered by releasing the ligand from the polymer and/or colonies may be mechanically (e.g., pipetted) removed.

In some embodiments, the present invention may be used to separate viral antigen-specific memory B cells from human peripheral blood mononuclear cells (PBMCs) derived from a vaccinated donor by indirect affinity magnetic bead methods. Materials and methods of the invention may include antigen conjugated biopolymer surfaces that can be used to mediate antigen specific B cell separation by differential rolling adhesion. In some embodiments, the present invention provides antigen conjugated biopolymer surfaces for antigen specific B cell separation. Experiments with antibody-conjugated microspheres will be used to evaluate surface gradients, shear rates, and flow cell design prior to experiments with B cells.

In some embodiments, B cells isolated using the present invention may be activated in situ for clone isolation. For example, biopolymer surface-captured antigen-specific B cells can be activated in situ for clone isolation.

In some embodiments, the present may be used to isolate antigen-specific B cells which can then be activated, induced to differentiate to antibody secreting cells (ASCs), and induced to expand and form colonies on the biopolymer surface. Colonies of activated antigen-specific memory B cells can be isolated from a biopolymer surface to generate IgG-secreting cell lines. Thus, the present invention provides materials and methods that may be used to isolate, expand, and characterize IgG-expressing cell lines derived from activated antigen-specific B cell colonies and generate clonal antibody-secreting cell lines.

### BRIEF DESCRIPTION OF THE DRAWINGS

FIG. 1 is a plasmid map of pcdna3.1zeo:VACV_B5R, pcdna3.1zeo:VACV_A33R, and pcdna3.1zeo:VACV_L1R.

FIGS. 2A and 2B are graphs of the antibody titre of circulating VACV-specific antibodies and B cells.

FIG. 3 shows the results of ELISA assays of human sera samples from Dryvax (LB,DS) or ACAM2000 (AM) vaccinated donors on recombinantly expressed VACV proteins with immunological relevance.

FIG. 4 shows the results of a PCR colony analysis of 10 XL-1 blue/pCOMB:HC+LC κ library clones at vector:insert ratios of 1:1 (top panel) or 1:5 (bottom panel) for presence of a cloned human LC κ cDNA insert.

FIG. 5 shows results of phage ELISA from different rounds of panning against L1 and MBP.

FIG. 6 is a photograph of an agarose gel picture and plasmid maps of pcDNAΔ:dhfr:VL:VH depicting two possible orientations for light and heavy chain cassettes.

FIG. 7 shows the results of an immunoprecipitation and polyacrylamide gel electrophoresis of radiolabeled VACV proteins with VACV-immune human sera or with mammalian cell-expressed full-length human mAbs.

FIG. 8 shows the protection of BALB/c mice by monoclonal antibody.

FIG. 9 shows weight change after i/n challenge with 2×10⁶ or 2×10⁵ PFU Vaccinia Virus IHD-J. Strain. Average weight of each treatment group (n=5) are shown.

FIG. 10 shows the design of a 12-zone microfluidic surface. Each zone may comprise a rectangular gold electrode to permit site specific addressing of functionally-conjugated chitosan mixtures.

FIG. 11 shows the design of the microfluidics flow chamber detailing the location of the electrode for electrodepostion of biofunctionalized surface and flow paths.

FIG. 12 shows a diagram of conjugated antigen mediated memory B cell separation and soluble ligand activation strategy.

FIG. 13 shows FACS data from PBMC thaw (Plot on left is in PBS alone and plot on right is PBS plus PI).

FIG. 14 shows the VACV L1 variants with multiple N- and C-terminal tags.

FIG. 15 shows agarose gels of (A) PCR product of L1R ectodomain and (B) PCR products of six terminally tagged variants of L1R.

FIG. 16 shows an agarose gel of Hind III and Not I restricted mammalian expression plasmid pcdna3.1+zeo:intA:VACVsL1R+His#1.

FIG. 17 shows an agarose gel of Hind III and Not I restricted mammalian expression plasmids containing six L1 terminally tagged variants.

FIG. 18 shows an SDS-PAGE gel of transiently expressed purified L1 variants.

FIG. 19 shows the results of an ELISA analysis of transiently expressed purified L1 variants.

### DETAILED DESCRIPTION OF THE INVENTION

The present inventor has been developing a monoclonal antibody cocktail for prophylactic and therapeutic treatment of pathogenic orthopoxvirus infection in humans. These infections may stem from adverse vaccination events (e.g., vaccinia virus (VACV) infections) or from exposure to other orthopoxviruses such as variola virus (VARV), the etiological agent for smallpox, or monkeypox virus (MPXV). The development of an effective therapeutic for these indications meets critical unmet medical and national security needs in the context of bioterror and biowarfare threats and public health challenges.

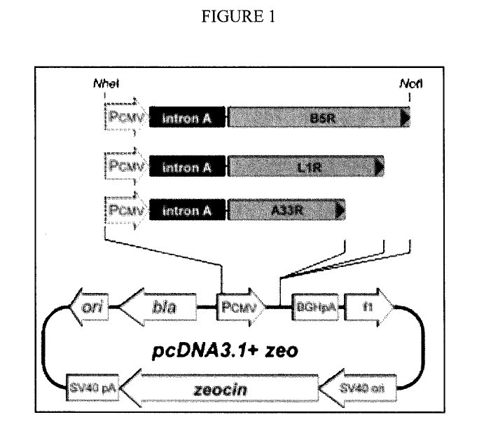

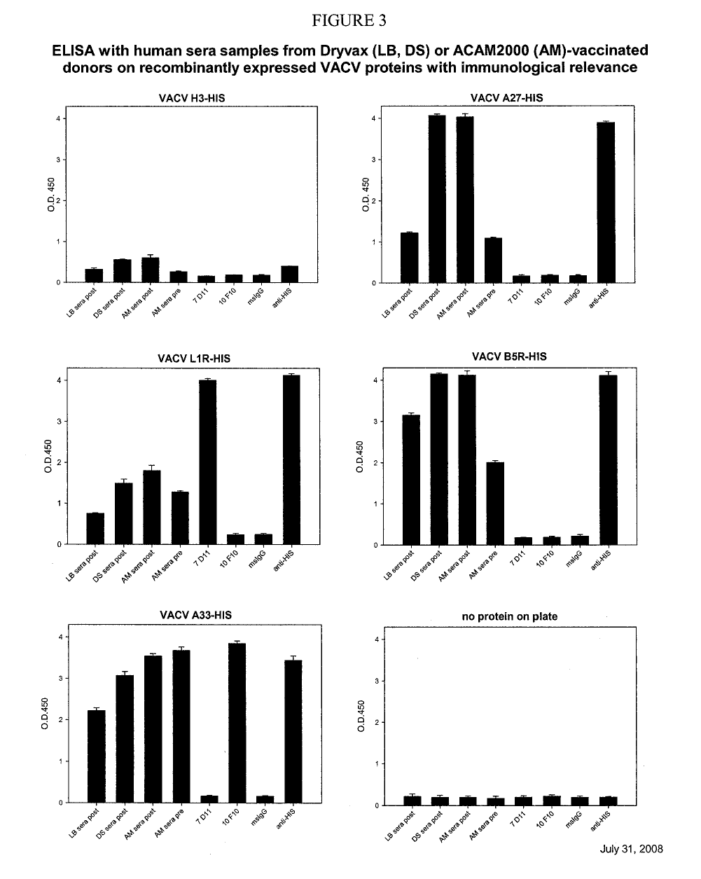

Efforts are focused on discovering mAb clones with specificities to VACV B5R, A33R, and L1R proteins, which are known to elicit neutralizing responses. Plasmids were engineered with these VACV genes in a pcdna3.1zeo plasmid background for generation of NS0 and 293T/17 cell lines expressing these anchored proteins on the cell surfaces (FIG. 1). In addition, expression vectors were constructed encoding HIS-tagged soluble versions of the following VACV proteins: A27L, D8L, A33R, B5L, A17L, A4L, A10L, H3L, F13L, and L1R. One skilled in the art will appreciate that other antigens, e.g., that are recognized by neutralizing antibodies, may be used in the practice of the present invention. TRIzol extracts of Vero cells infected with VACV strain WR were prepared for isolation of total RNA and cDNA synthesis. These cDNAs were used to amplify all 10 viral genes indicated above. Soluble versions of each gene were engineered to contain only the ectodomain tagged to a poly-histidine tag and are driven by the VACV B5 signal peptide sequence. Expression of HIS-tagged soluble versions of VACV proteins A33, B5, and L1 was attempted in both CHO-S and 293-F FreeStyle cells (Invitrogen). Cells were transfected according to manufacturer's instructions, and each was allowed to proceed under growth conditions for 96 hours. For initial purification, supernatants were applied to an equilibrated HIS Spin column (Sigma), washed, and eluted with 100 mL of buffered saline buffer containing imidazole. As shown in FIG. 3, both CHO-S and 293-F cells expressed sB5-HIS and s-L1-HIS, but neither cell line expressed significant levels of sA33-HIS. Yields of sB5-HIS and s-L1-HIS ranged between 0.5 to 34 μg/mL supernatant. Thus, we employed CHO-S and/or 293-F cells to generate sB5-HIS and sL1-HIS, and 293T/17 cells grown in serum containing medium to generate sA33-HIS.

FIG. 1 shows a plasmid map of pcdna3.1zeo:VACV_B5R, pcdna3.1zeo:VACV_A33R, and pcdna3.1zeo:VACV_L1R. A NheI-NotI fragment comprising the majority of the pCMV promoter, including intron A, and the complete open reading frame of VACV A33R, B5R, or L1R was cloned into a pcdna3.1zeo plasmid fragment restricted with the same endonucleases. The resulting constructs were used to transfect 293T/17 cells for confirmation of expression, and for subsequent generation of stable lines expressing these viral genes on the cell surface.

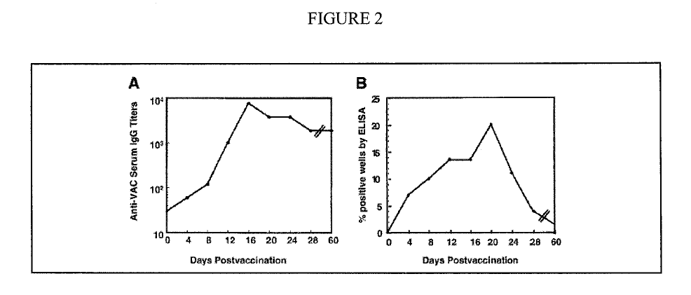

Human Fab phage libraries were constructed with cDNAs generated from peripheral blood lymphocytes (PBLs) of VACV-immunized human donors. Isolation of PBLs from the boosted donors was conducted on Day 18 post-vaccination correlating with the peak of the humoral immune response (see FIG. 2). A VACV-immune volunteer was boosted with the licensed New York Board of Health VACV vaccine. Circulating anti-VACV IgG was measured by ELISA at various times after vaccination. B cells were transformed with Epstein-Barr virus and limiting dilutions were added to 96-well plates. The frequency of circulating B cells secreting antibodies to VACV was estimated by ELISA of wells containing cell clones.

During the construction of the first human Fab phage display library from donor DS, expression and purification of recombinant VACV protein targets was progressing in parallel. Upon availability of these reagents, it was determined that serum from donor DS was poorly reactive to VACV L1, a critical specificity. A second donor, AM, was vaccinated with ACAM2000 (Acambis) and PBLs were isolated. Serum from both donors were analyzed by ELISA for reactivity to a panel of recombinant VACV protein targets. As shown in FIG. 3 below, all sera and positive control (anti-HIS mAb) reacted poorly with sH3-HIS protein. The cause for the lack of significant reactivity is not presently known. There is a possibility that although the protein was purified and quantified that it is misfolded and non-reactive. Conversely, reactivity to all other proteins was clearly established with the anti-HIS positive control mAb. In addition, 7D11 reacted only with sL1-HIS, and 10F10 reacted only with sA33-HIS, as expected. Serum from donor AM showed the best reactivity to all antigens, and RNA isolated from this donor's PBLs was subsequently used for human Fab library construction. In panel VACV sL1R-HIS, serum from donor “DS sera post” was used at 1:10 dilution, as poor reactivity had been seen with this donor's sera in previous experiments when used at 1:100 dilution.

Using the PBMCs derived from Donor AM, kappa and lambda Fab phage display libraries were created with the pCOMB3 system (Scripps Institute)². Cells were subjected to TRIzol (Invitrogen) treatment to isolate total RNA. The TRIzol prep was used to generate a cDNA library derived from the mRNAs using RT-PCR (Invitrogen, SuperScript III). The resulting cDNA library was used as a template to PCR amplify variable heavy (VH) and light chain (VL κ and VL λ) regions using broad coverage primers sets. In order to optimize efficiency of the small-scale ligation of VL κ into pCOMB3:VH prior to commencing large-scale transformations, the PCR products from 9 independent VL κ cDNA specificities were pooled, then restricted with SacI and XbaI. The resulting fragments were cloned into the similarly restricted phagemid vector pCOMB3 containing the human VH (designated pCOMB3:VH). To determine library size and transformation efficiency on a small-scale, test ligations were performed followed by electroporation into electrocompetent E. coli XL-1 blue cells (Stratagene). To determine the optimal ratio of pCOMB3:VH vector to VL κ insert, four transformations were performed using 250 ng vector with the following vector:insert ratios; (1) 1:1, (2) 1:5, (3) 1:10, (4) 1:15. Table 1 outlines the titer calculations and the resulting library size for each transformation.

TABLE 1
pCOMB3:VH + VL κ small-scale library titer calculations

| Vector: | | | | |
|---|---|---|---|---|
| Insert | Volume | # | | Titer |
| ratio | plated | colonies | Conversion calculation | (cfu/ug DNA) |
| 1:1 | 100 μL | 482 | 4.82/μL × 1000 μL/ | 2.514e5 |
| | | | mL × 13.04 mL | |
| 1:5 | 100 μL | 459 | 4.59/μL × 1000 μL/ | 2.394e5 |
| | | | mL × 13.04 mL | |
| 1:10 | 100 μL | 329 | 3.29/μL × 1000 μL/ | 1.716e5 |
| | | | mL × 13.04 mL | |
| 1:15 | 100 μL | 305 | 3.05/μL × 1000 μL/ | 1.591e5 |
| | | | mL × 13.04 mL | |

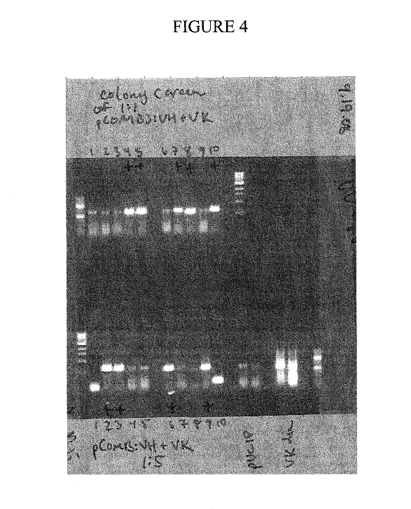

Since the optimal vector:insert ratio for the pCOMB:VH+VL λ was determined to be 1:1, initial PCR analysis to determine the percentage of clones incorporating a VL κ insert focused on the 1:1 and 1:5 ratio ligation reactions since the VL κ is identical to VL λ in size and in cloning methodology. Ten clones from the 1:1 and 1:5 ligation reactions were chosen at random from LB Carb100 plates and were subjected to colony PCR analysis using a human light chain Fab specific primer (CK2d) and a mix of the 9 VL κ oligonucleotides used to amplify the 9 independent VL κ cDNAs. Amplified PCR products were analyzed by agarose gel electrophoresis. A DNA band of ca. 700 bp is indicative of a cloned VL λ cDNA insert in pCOMB3:HC amplified with a mix of VL κ oligonucleotides and CK2d. The negative control pUC18 did not amplify as expected. The positive control, a diluted PCR product from the initial VL κ PCR amplification, produced the expected band at ca. 700 bp. As shown in FIG. 4, 5/10 and 4/10 clones analyzed from the 1:1 and 1:5 ligation ratios, respectively, showed the presence of a light chain κ cDNA insert (ca. 700 bp) indicating that 50% of the 1:1 ratio plate and 40% of the 1:5 ratio plate were positive for the LC κ insert. Subsequent cloning of the large-scale human VH library and insertion of the VL κ was performed at the 1:1 ratio to achieve the highest transformation efficiency and percentage of positive clones.

Prior to commencing large-scale ligations and transformations of the dual human chain Fab κ and λ libraries, a stock of helper phage VCSM13 (Stratagene) was generated to facilitate phage production from the phagemid pCOMB3 backbone which, except for the origin of replication of the filamentous bacteriophage f1, lacks the necessary genes for replication and assembly of phage particles. Helper phage VCSM13 interacts with the phagemid genome by providing the necessary replication and assembly proteins required for phagemid packaging and display of the pIII-fused Fab proteins on the phage surface. Multiple large-scale ligations and transformations of SacI and XbaI restricted VL λ insert into the similarly restricted pCOMB3:VH vector in a 1:1 ratio were performed to generate a library size approaching the desired range of 10⁷-10⁸ individual transformants and to compensate for the ˜50% LC λ insertion percentage. Six transformations were performed with 300 μL electrocompetent XL-1 blue cells, each with DNAs from identical ligation reactions. Ten, 1, and 0.1 μL aliquots from each transformation were plated on LB Carb100 agar plates to titer the transformants, following the addition of 10 mL SB medium (Carb 20 μg/mL, tetracycline 10 μg/mL). The cultures were incubated at 37° C. for 1 hr with shaking. The carbenicillin concentration was adjusted to 50 μg/mL followed by an additional 1 hr incubation at 37° C. with shaking. The cultures were superinfected with 1 mL VCSM13 helper phage (˜10¹² pfu/ml), transferred to flasks containing 100 mL SB (Carb50, Tet10) medium, and incubated at 37° C. with shaking. After 2 hours, kanamycin (70 μg/mL) was added prior to overnight incubation. The following day, the E. coli cells were pelleted by centrifugation at 3000 rpm, 4° C., for 15 min. Pellets were harvested for future DNA preparation. The resulting supernatant was transferred to a fresh tube and 4% (w/v) PEG-8000 and 3% (w/v) NaCl was added to precipitate the phage. The supernatant was incubated on ice for 30 min prior to centrifugation at 9000 rpm, 4° C., for 20 min. The packaged phagemid pellet was resuspended (2 mL TBS/1% BSA) then stored at 4° C. This procedure was repeated to generate the dual chain human Fab κ combinatorial phage library.

To titer the λ combinatorial phage library, colonies emerging from the LB Carb100 plates outlined above were counted and the overall size of the library from six transformations was back calculated. Table 2 outlines titer calculations and the resulting library size for each transformation, as well as the combined library titer. Twenty-one clones chosen at random from 6 independent LB Carb100 plates were subjected to colony PCR analysis using a human heavy chain Fab specific primer (CG1d) and a pCOMB3 specific primer (VH#2). Amplified PCR products were analyzed by agarose gel electrophoresis. Nineteen of 21 clones or 90.5% analyzed showed the presence of a heavy chain cDNA insert (ca. 700 bp). Twenty additional clones, chosen at random, were subjected to colony PCR analysis using a human light chain Fab primer (CK2d) and a mix of the 12 VL oligonucleotides used to amplify the 12 independent VL λ cDNAs. Eighteen of 20 clones or 90% analyzed contained the VL λ insert (ca. 700 bp). These combined results provide substantial confidence that approximately 81.4% of the library clones contain a relevant HC and LC λ insert. The corrected library size can, therefore, be equated to 81.4% of the originally calculated titer, or 9.418×10⁶ independent clones.

To titer the κ combinatorial phage library, colonies emerging from the LB Carb100 plates outlined above were counted and the overall size of the library from six transformations was back calculated. Table 3 outlines titer calculations and the resulting library size for each transformation, as well as the combined library titer. Twenty-two clones chosen at random from 6 independent LB Carb100 plates were subjected to colony PCR analysis using a human heavy chain Fab specific primer (CG1d) and a pCOMB3 specific primer (VH#2). Amplified PCR products were analyzed by agarose gel electrophoresis. Fourteen of 22 clones or 63.6% analyzed showed the presence of a heavy chain cDNA insert (ca. 700 bp). Twenty additional clones, chosen at random, were subjected to colony PCR analysis using a human light chain Fab primer (CK2d) and a mix of the 9 VL κ oligonucleotides used to amplify the 9 independent VL κ cDNAs. Fourteen of 20 clones or 70.0% analyzed contained the VL κ insert (ca. 700 bp). These combined results provide substantial confidence that approximately 44.5% of the library clones contain a relevant HC insert. The corrected library size can, therefore, be equated to 44.5% of the originally calculated titer, or 2.370×10⁶ independent clones.

TABLE 2
Large-scale pCOMB3:VH:VL λ library titer calculations

| Plate | | # | | Titer |
|---|---|---|---|---|
| # | Volume | colonies | Conversion calculation | (cfu/ug DNA) |
| 1 | 0.1 μL | 15 | 150/μL × 1000 μL/ | 1.995 × 10⁶ |
| | | | mL × 13.3 mL | |
| 2 | 0.1 μL | 8 | 80/μL × 1000 μL/ | 1.064 × 10⁶ |
| | | | mL × 13.3 mL | |
| 3 | 0.1 μL | 14 | 140/μL × 1000 μL/ | 1.862 × 10⁶ |
| | | | mL × 13.3 mL | |
| 4 | 0.1 μL | 14 | 140/μL × 1000 μL/ | 1.862 × 10⁶ |
| | | | mL × 13.3 mL | |
| 5 | 0.1 μL | 28 | 280/μL × 1000 μL/ | 3.724 × 10⁶ |
| | | | mL × 13.3 mL | |
| 6 | 0.1 μL | 8 | 80/μL × 1000 μL/ | 1.064 × 10⁶ |
| | | | mL × 13.3 mL | |
| Total | | | | 1.157 × 10⁷ |
| | | | Corrected Library | 9.418 × 10⁶ |
| | | | Size (95.8%) | |

TABLE 3
Large-scale pCOMB3:VH:VL κ library titer calculations

| Plate | | # | | Titer |
|---|---|---|---|---|
| # | Volume | colonies | Conversion calculation | (cfu/μg DNA) |
| 1 | 0.1 μL | 7 | 70/μL × 1000 μL/ | 9.310 × 10⁵ |
| | | | mL × 13.3 mL | |
| 2 | 0.1 μL | 14 | 140/μL × 1000 μL/ | 1.862 × 10⁶ |
| | | | mL × 13.3 mL | |
| 3 | 0.1 μL | 7 | 70/μL × 1000 μL/ | 9.310 × 10⁵ |
| | | | mL × 13.3 mL | |
| 4 | 0.1 μL | 3 | 30/μL × 1000 μL/ | 3.990 × 10⁵ |
| | | | mL × 13.3 mL | |
| 5 | 0.1 μL | 7 | 70/μL × 1000 μL/ | 9.310 × 10⁵ |
| | | | mL × 13.3 mL | |
| 6 | 0.1 μL | 2 | 20/μL × 1000 μL/ | 2.660 × 10⁵ |
| | | | mL × 13.3 mL | |
| Total | | | | 5.320 × 10⁶ |
| | | | Corrected Library | 2.370 × 10⁶ |
| | | | Size (95.8%) | |

To identify human Fabs specific for VACV proteins with immunological relevance, the dual combinatorial pCOMB3:VH:VL κ library was panned against immobilized, his-tagged L1. Generating specific phage binders to a specific antigen, such as L1, involves multiple rounds of panning the phage pool to an antigen immobilized on an ELISA plate. During each round, the phage clones that specifically bind to the antigen are enriched while the amount of non-specific binders are reduced by increasing the washing stringency. Briefly, 1 μg of recombinant soluble HIS-tagged L1 was diluted in 25 μl of 0.1 M sodium bicarbonate (pH 8.6) and incubated in a 96-half well ELISA plate overnight at 4° C. The following day, after removal of the coating solution, the well was blocked with 150 μl of 3% (w/v) BSA in TBS, and incubated at 37° C. for 2 hours. Fifty microliters of the dual κ phage library, outlined above, was then added to the ELISA plate after removal of the blocking solution and was incubated at 37° C. Following a 2 hour incubation, the phage solution was removed and the well was washed by adding 150 μl of 0.5% (v/v) Tween20 in TBS, pipeting 5 times vigorously, then incubating for 5 minutes. During the first, second, third, and fourth rounds of panning, this washing process was repeated 1, 5, 10, and 15 times, respectively. To elute the L1-specifically bound phage, 50 μl of 100 mM glycine-HCl (pH 2.2) was added to the well, followed by a 10 minute incubation at room temperature. After vigorous pipetting, the phage were removed from the well and incubated with 3 μl of a 2M Tris-base solution to neutralize the acidic elution buffer. The neutralized phage eluate was added to a 2 ml XL1-blue E. coli culture grown in SB (Tet¹⁰) to an O.D. of A₆₀₀=1.0, then incubated at room temperature for 15 minutes. The resulting infected culture was titered, reamplified as above, and the precipitated phage preparation was panned a consecutive time against L1. This procedure was repeated through four rounds of panning.

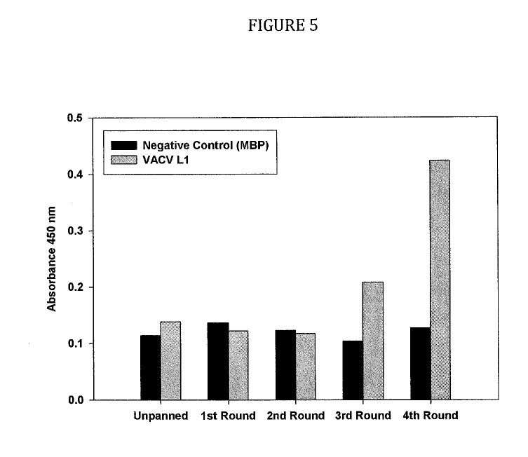

To assess the success of the panning experiment, the phage pools generated during each round of panning were tested by ELISA to determine the specificity to L1 using an anti-M13 detection reagent. The results of the panning experiment are illustrated in FIG. 5. As expected, the phage preparations from each round of panning reacted similarly for the negative control antigen MBP with absorbance readings comparable to background. The phage preparations also reacted with the positive control antigen, to a similar degree, detecting non-specific Fab-expressing phage resulting in absorbance values ranging from 1.060 through 1.218 (data not shown). Specific binding of the phage preparations to the target antigen VACV L1 increased over the panning rounds, especially during rounds three and four, suggesting L1 specific binders were selected and amplified.

To identify single clones expressing Fabs specific to L1, individual colonies from the output titer plates outlined above from the fourth round of panning were screened. One milliliter cultures inoculated with a single colony were grown overnight in SB/Carb⁵⁰ broth at 37° C. with shaking. The following day, 2 μl of the overnight culture was spotted on a LB Carb100 plate, 100 of the overnight culture was used to seed a 5 ml SB/Carb⁵⁰ culture corresponding to the same clone, and the remaining volume from the overnight culture was preserved for future DNA preparation. The 5 ml culture was grown to an OD₆₀₀=˜0.6 and was subsequently induced with 1 mM Isopropyl β-D-1-thiogalactopyranoside (IPTG). The induced cultures were incubated overnight at 30° C. with shaking. The next day, each clone was centrifuged at 2500×g for 20 minutes at 4° C. The cell pellet was resuspended in 500 μl PBS then subjected to 4 cycles of 5 minutes in a dry ice/ethanol bath followed by 5 minutes at 37° C. to lyse the cells. The samples were centrifuged at 15,000 rpm to pellet cellular debris. The Fab-containing supernatants harvested from cell lysates were transferred to fresh tubes prior to analysis.

Over the course of one month, a total of 1370 clones generated from the human kappa combinatorial library were evaluated by the methods outlined above. Of the 1370 individual clones screened, only 165 or 12% were positive for non-specific Fab production; none of which were specific for L1. The low percentage of colonies not expressing Fab could be explained by an initial over-estimation of clones that contain both a HC and LC insert; a theoretical calculation based on only 20 clones had been screened by PCR for HC or LC insertion. Also, the first generation pCOMB3 vector that was used to construct this library construction contains repeated lac promotor and pelB leader sequences that may result in stability problems in the resulting colonies. In addition, the repetitive nature of the phagemid promoter and leader sequences facilitates recombination events that can lead to insert deletion. Smaller phagemids lacking HC and/or LC inserts may have been more efficiently packaged than the larger phagemids containing both inserts and, therefore, despite stringent washing steps during panning, these non-expressing clones may have been more prominent in the output titer plates used for individual colony Fab expression analysis. Many of the non-expressing Fab clones may have contained only a HC or LC or neither. Therefore, the low percentage of clones expressing functional Fabs is most likely due to poor insertion rates of HC and LC during the initial, sequential transformations in combination with a high likelihood of recombination events due to repeated sequences in the pCOMB3 vector itself.

To combat the molecular cloning issues outlined above, efforts were shifted to construct a new dual human Fab combinatorial library using the same individual VH and VL regions amplified for this present library, but instead of sequential VH and VL ligations and transformations, the variable regions were linked through a series of PCR steps yielding one final Fab product used to clone into the phagemid backbone via a single ligation and transformation. Also, a second-generation pCOMB vector, specifically pCOMB3X, was utilized as the phagemid vector backbone. pCOMB3X is a more suitable vector over pCOMB3 since the phagemid pCOMB3X contains a single lac promotor and uses a combination of ompA and pelB leader sequences as opposed to the dual pelB sequences of pCOMB3 which more readily facilitate recombination events. Constructing a new dual library with a single ligation/transformation step and using a present-generation pCOMB3X vector was expected to yield a higher percentage of overall clones containing both a VH and VL insert with increasing stability.

Since the pCOMB3X vector backbone maintains identical VH and VL cloning sites used for the first generation pCOMB3 vector yet incorporates separate leader sequences for the heavy chain and light chain (pelB and ompA, respectively), the final library construction approach was to clone the VH and VL regions into the present generation pCOMB3X sequentially, instead of as one unit. This was expected to dramatically decrease recombination events that occurred during the initial library construction effort where both the heavy and light chains were under control of the same pelB leader sequence.

To insert the heavy chain pool into the pCOMB3X:light chain combinatorial library, the HC PCR products and pCOMB3X:light chain DNA were restricted with XhoI/SpeI, ligated, and transformed into XL1-blue E. coli cells, as described previously. Briefly, an initial test ligation of 140 ng of XhoI/SpeI restricted pCOMB3X:LC was mixed with the similarly restricted HC pools at a vector:insert ratio of 1:3 and ligated overnight. An identical reaction containing only the vector was used to determine the percentage of background. One hundred, ten, and one μL aliquots from each transformation were plated on LB Carb100 agar plates, and colonies were subsequently counted then the transformation efficiency was backcalculated. Unfortunately, the difference in total number of colonies between the pCOMB3X:LC+HC reaction and the background plate with vector only was negligible. This comparable transformation efficiency is most likely due to insufficient restriction of the pCOMB3X:LC vector, which would cause high background due to self-ligation of the vector and not permit successful cloning of the HC insert. As expected, 20/20 clones that were subjected to colony PCR analysis from the test ligation plate were negative for the HC insert.

To combat the lack of HC insertion into the phagemid vector, the pCOMB3X:LC combinatorial library was restricted with XhoI/SpeI for 8 hours, gel purified, then restricted a second time with XhoI/SpeI for an additional 8 hours. The pCOMB3X vector contains a substantial stuffer fragment between the XhoI and SpeI sites that should facilitate efficient restriction. In addition, the XhoI and SpeI cohesive end sites should permit directional cloning and not easily self-ligate. Nevertheless, the XhoI/SpeI double restricted LC phagemid vector was treated with antarctic phosphatase to remove the 5′ phosphate groups and prevent self-ligation. A second round of test ligations and transformations inserting the HC into the pCOMB3X:light chain combinatorial library was performed, as outlined above. Despite extensive efforts to hinder self-ligation of the vector backbone, the difference in total number of colonies between the pCOMB3X:LC+HC reaction and the background plate with vector only was again negligible. Upon colony PCR analysis, 1 of 20 clones were positive for the HC insert. Due to the significant cloning obstacles encountered and subsequent insufficient dual human Fab combinatorial library size, no further discovery work was pursued using pCOMB3 technology and approaches.

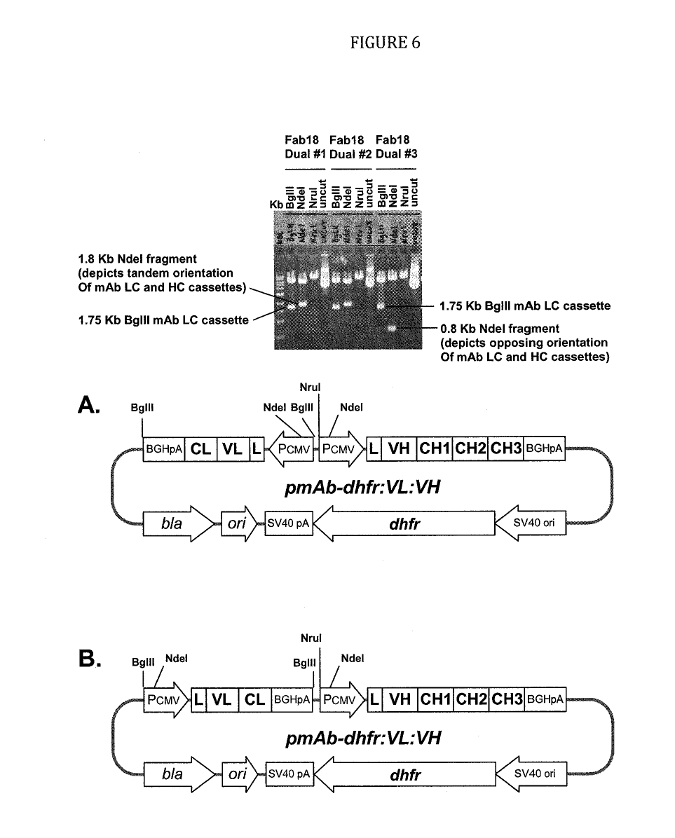

The mammalian expression vector pcdnaΔ:dhfr was constructed and served as the backbone for the cloning of mammalian genes, including human antibody heavy and light chain coding sequences, for expression in the dihydrofolate reductase (dhfr) mutant parental cell line CHO-DG44. Using this vector, a human IgG1 heavy chain constant region expression cassette was cloned in the multiple cloning site (mcs), and served as acceptor for human heavy chain variable sequences. This vector was designated pdhfr:Hcassette. Four constructs were generated that express full-length human IgG1 heavy chain for each of four monoclonal antibodies (mAbs) specific to poxvirus envelope proteins previously isolated from an Fab phage display library by United States Army Medical Research Institute of Infectious Diseases (USAMRIID) researchers³. The corresponding light chain variable sequences were first cloned into a baculovirus expression vector containing a human light chain constant region cassette, pIEI-light. The entire light chain coding sequence was subsequently cloned into pcdnaΔ:dhfr. To construct a dual expression vector, primers specific to the 5′ end of pCMV-MIE and to the BGH-pA on the complementary strand were used to amplify a light chain cassette containing pCMV-MIE—light chain—BGHpA, which contained BglII REN sites on both ends. This fragment was subsequently cloned into the unique BglII site in each corresponding pdhfr:Heavy_Chain construct. The resulting construct contained complete human antibody light and heavy chain genes cloned in tandem. Dual constructs were used to generate stable mammalian cell lines for large-scale production of mAbs. Four monoclonal antibody dual expression constructs were generated, and were designated mAb 14, mAb 18, mAb 19, and mAb 21, to keep with historical nomenclature. The two possible orientations for full length light and heavy antibody gene sequences in the pcDNAΔ:dhfr expression vector are depicted in FIG. 6. Cloning of the LC cassette into the unique BelII site located between the bla gene and the pCMV promoter cassette produces two possible orientations: LC and HC cassettes in tandem, or in opposing orientations. Restriction of the constructs with BglII produces a single insert band of 1.75 Kb, corresponding to the light chain cassette, in addition to a 6.5 Kb vector band. NruI restricts the entire construct once, between the bla gene and the pCMV promoter cassette in the vector sequence. Restriction with NdeI, which cuts once in the pCMV promoter cassette results in two possible fragments: a 0.8 Kb fragment indicates cloning of the LC and HC cassettes in opposing orientations, as depicted in plasmid map “A” below. In contrast, a 1.8 Kb fragment indicates cloning of the mAb genes in tandem, as depicted in plasmid map “B”. Expression of mAb from each of the four dual constructs generated were tested transiently in human endothelial kidney cells (HEK-293T) before generation of stable mammalian cell lines. Transfection supernatants were assayed for presence of a full length human IgG by capture ELISA. The supernatants were also assayed for mAb binding specificity in a VACV-ELISA at USAMRIID.

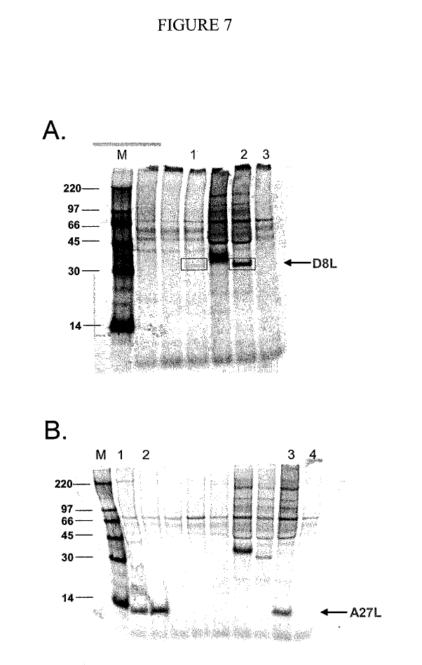

The full coding region of the antibody light and heavy chains were determined by DNA sequencing. The correct sequence for each light and heavy chain has been confirmed for clones 14 and 21 by comparing with previously determined sequences for these genes. mAb 14 binds specifically to VACV D8L, and mAb 21 binds to A27L. Clones 18 and 19 yielded variable domains with amino acid sequences different from those previously determined for these clone designations. Clones 18 and 19 were supposedly specific for B5R and L1R. mAbs 18 and 19 were not pursued based on their inability to immunoprecipitate proteins from radiolabeled COS-7L cells transfected with B5R and L1R expression constructs. Immunoprecipitation of VACV D8L and A27L proteins with mAbs 14 and 21, respectively, is depicted in FIG. 7. COS cells transfected with recombinant pWRG7077 DNA vector expressing VACV D8L or A27L protein were radiolabeled with ³⁵S-cysteine and -methionine. Cell lysates were prepared in 4% Zwittergent lysis buffer, and radiolabeled proteins were immunoprecipitated using protein G agarose beads, as previously described (Guttieri et al., 2003). Panel A: VACV D8L protein was immunoprecipitated from transfected cell lysates using mAb14 (Lane 1) and VACV-immune human sera (Lane 2, positive control). As a negative control, VACV-immune human sera was used to immunoprecipitate proteins from radiolabeled cell lysates transfected with empty vector pWRG7077 (Lane 3). Panel B: VACV A27L protein was immunoprecipitated from transfected cell lysates using mAb 21 (Lanes 1 and 2) and VACV-immune human sera (Lane 3, positive control). As a negative control, VACV-immune human sera was used to immunoprecipitate protein from radiolabeled cell lysates transfected with empty vector pWRG7077 (Lane 4). The sizes of molecular weight markers (M) are shown to the left of each panel, and VACV D8L and A27L proteins are indicated. The location of VACV D8L protein in Panel A is outlined in red.

The nucleotide sequence of the dhfr selectable marker was confirmed, and transfection and selection studies demonstrated that the cloned dhfr gene in pcdnaΔ:dhfr is functional. Upon confirmation of mAb sequences for each construct, stable mammalian cell lines were generated for clones 14 and 21, using the dhfr(−) background cell line CHO-DG44. Transfections were selected in varying concentrations of the selective inhibitor methatrexate (MTX), and emerging stable cell isolates were assayed for specific productivity of human IgG1. Initial characterization was performed on greater than 400 wells for each of the stable cell lines. Isolates expressing significant levels of human IgG1 were expanded to larger well plates, followed by small T flasks, and finally to medium T flasks. When cells reached greater than 70% confluence in medium T flasks the initial specific productivity rate (SPR) characterization of relevant clones commenced. Nineteen CHO:mAb14 and sixteen CHO:mAb21 isolates were characterized by SPR. The cell lines with the best specific productivity from each lineage were subjected to MTX amplification. Four CHO:mAb14 and seven CHO:mAb21 cell lines amplified with MTX were subjected to a first round of SPRs. CHO:mAb14 isolate 14.4.200 and CHO:mAb21 isolates 21.27.200 and 21.157.200, which appear to have undergone amplification with MTX, as determined by higher productivity rates than the unamplified counterparts, were cultured in CHO-S-SFM II. This serum-free medium was used to produce small quantities of mAbs 14 and 21 for IP and neutralization assays.

To test the ability of mAbs 14 and 21 to reduce infectivity of VACV strain WR on VeroE6 cell monolayers, plaque reduction neutralization test (PRNT) assays were performed with both antibodies, each purified from transient HEK-293T or stable CHO cultures. Each test was performed in duplicate, and results are averages of results obtained for each tested mAb concentration. VACV PRNT were performed essentially as described by Schmaljohn et al (1999). Both mAbs 14 and 21 showed significant activity in a PRNT assay.

Subsequently, supernatants from single CHO-DG44:mAb 14 and 21 cell lines growing in CHO-SFM-II were subjected to Protein-A purification to isolate small quantities of purified mAb for functional studies. These purified mAbs were used in a round of plaque neutralization assays. Both mAbs 14 and 21 showed significant activity in a plaque reduction and neutralization assay. Further, mAbs generated in stable CHO cell lines and purified from spent SFM cultures performed better in PRNT assays than counterparts produced in transient HEK-293T cultures in serum supplemented media. Results from this experiment are shown in Table 4.

MAbs were purified from single CHO-DG44:mAb 14 and 21 cell lines growing in CHO-SFM. Culture supernatants were subjected to Protein-A purification and human IgG was eluted, concentrated and quantitated by Bradford Assay and human IgG ELISA. Quantitated IgGs were used in a standard PRNT assay at USAMRIID A purified human IgG1 was purchased from Sigma and used as a negative control in PRNT assays. Data is the average of duplicate wells for each condition tested. The results are shown in Table 4. Table 4 gives the percent VACV plaque neutralization with mAbs 14 (α-D8L) and 21 (α-A27L) compared to control human IgG1.

TABLE 4

| | % neutralization compared to empty | | | | |
|---|---|---|---|---|---|
| | vector ctrl | | | | |
| | amount of mAb per well (μg) | | | Controls | |
| monoclonal | 6 | 3 | 1.5 | Empty | n/a |
| antibody | | | | vector | |
| mAb 14 | 87.0 | 79.4 | 74.1 | (+) | 92 |
| | | | | sera @ | |
| | | | | 1:100 | |
| mAb 21 | 82.6 | 58.8 | 37.0 | Virus | n/a |
| | | | | alone | |

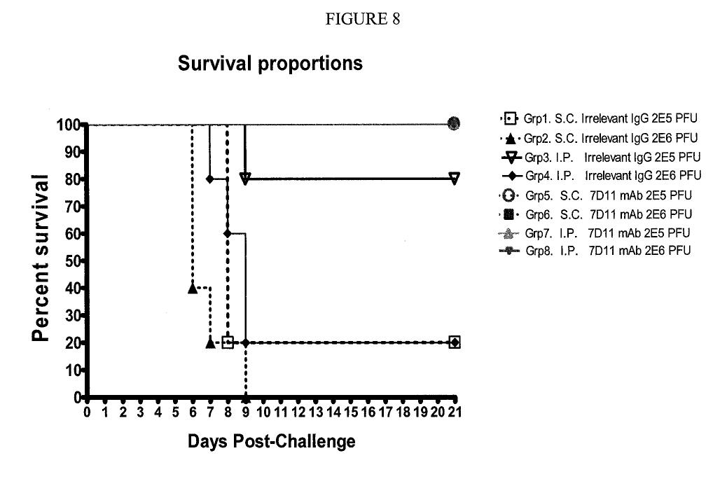

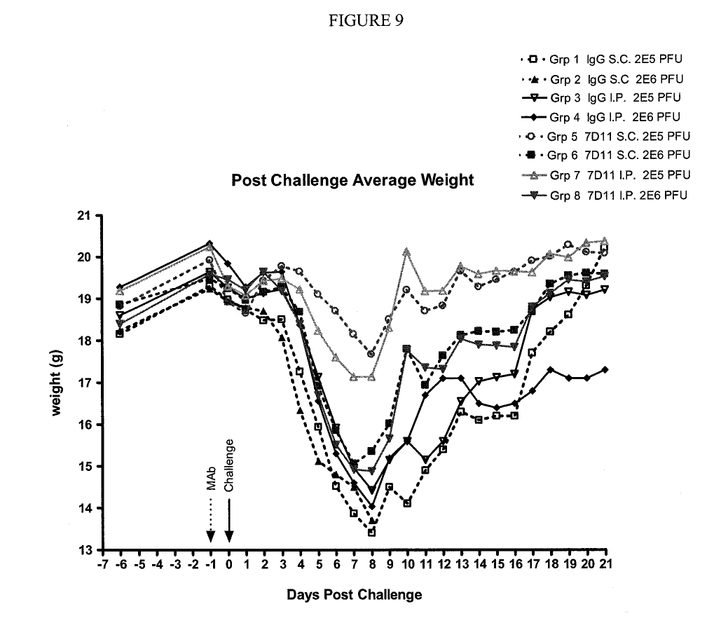

The objective of a follow-on study was to validate an antibody passive transfer model of protection of BALB/c mice against lethal VACV intranasal challenge. The study assessed survival of two challenging dose groups (2×10⁶ and 2×10⁵ PFU/mL) of VACV IHD-J and compared two antibody administration routes (subcutaneous and intra-peritoneal). The positive control used was a murine anti-VACV (L1 specific) monoclonal antibody, 7D11, known to protect mice in earlier studies. 7D11 and negative control human irrelevant IgG were passively administered by either i.p or s.c. routes and 24 hours later all animals were intranasally challenged with 2×10⁵ or 2×10⁶ PFU Vaccinia Virus IHD-J. Strain. Mice were euthanized when their weight fell below 70% of initial weight. The survival curve data in FIG. 8 indicates complete protection in all 7D11 treatment groups. By contrast, negative control groups demonstrated only 0˜20% survival, except group 3 (80% survival). In particular, 0% survival of negative control group 2 (Irrelevant IgG, s.c./10⁶ PFU challenged) relative to 100% survival of 7D11 treated group 6 (7D11, s.c./10⁶ PFU challenged) is highly significant. FIG. 9 shows the weight change after i/n challenge with 2×10⁶ or 2×10⁵ PFU Vaccinia Virus IHD-J. Strain. Average weight of each treatment group (n=5) are shown. The post challenge weight shown in FIG. 9 suggest significant weight loss in all negative control groups through day 4˜8 which correlate with survival data. The mean weight loss of negative control groups at day 8 ranged between 25%-30% compared to approximately 11%˜22% in 7D11 treated animals. Interestingly, similar weight loss/recovery pattern were observed in mice with 7D11 via s.c. or i.p. route, although mice in Groups 6 and 8 challenged with the higher dose of virus experienced a greater degree of weight loss between days 4-7 p.i. In this pilot study, complete protection was achieved by mouse 7D11 Mab. The objective of further work is to evaluate the ability of anti-VACV virus monoclonal antibodies mAb 14 and mAb 21 to protect BALB/c mice against lethal VACV intranasal challenge in a passive transfer study. Study groups will include 200 μg/animal individual mAb doses and two groups that investigate combinations of mAb 14 and 21 for synergistic protection.

Several opportunities exist to improve the current discovery and manufacturing approaches for therapeutic mAbs. Our prior results strongly suggest that the use of peripheral blood mononuclear cells (PBMCs) from immunized donors may provide a source of B cell clones that express B cell receptors (BCRs), in the case of memory B cells, or secrete IgG, in the case of antibody secreting cells, that neutralize virus. The current problem areas in the Fab phage display discovery strategies demonstrate issues related to the amplification and manipulation of nucleic acid sequences and the inherent instabilities of the phage display systems. An alternate approach that preserves the advantages of using the human donor B cells, yet avoids the problems associated with gene manipulation is to isolate the specific B cells of interest directly. The circulating pool of antigen-specific memory B cells may also convey the added value of the natural affinity maturation process that occurs during the late adaptive immune response.⁴⁻⁹ These circulating antigen-specific memory B cells may provide antibodies of the highest affinity and potency. In addition, an approach that results in manufacturing cell lines directly derived from the original human antigen-specific memory B cells through the processes of immortalization may provide significant advantages including cell line stability, antibody quality/fidelity, reduced speed to clinic, and lower cost of therapeutic development.¹⁰⁻¹³ Through the use of a biopolymer scaffold to present antigen and controlled microfluidics, B cells of interest may be captured, separated by affinity, activated, and cultured in a single device. As an added benefit, such a platform may be useful in the investigation of the human immune response.

The present invention provides materials and methods that all one skilled in the art to design, develop, and characterize a device for the isolation of antigen-specific memory B cells for the discovery and manufacture of therapeutic monoclonal antibodies.

It will be readily apparent to one of ordinary skill in the relevant arts that other suitable modifications and adaptations to the methods and applications described herein are obvious and may be made without departing from the scope of the invention or any embodiment thereof. Having now described the present invention in detail, the same will be more clearly understood by reference to the following examples, which are included herewith for purposes of illustration only and are not intended to be limiting of the invention.

Experiments were designed to demonstrate isolation of VACV L1-specific memory B cells from the same ACAM2000 vaccinated donor used to generate the Fab phage display library using recombinant L1 antigen. The donor was vaccinated approximately 6 months ago and, based on recent studies,¹⁴ should currently maintain on the order of 1% VACV-specific circulating memory B cells per total IgG⁺ circulating memory B cells. It has been established that IgG⁺ memory B cells comprise 10-15% of the peripheral B cell compartment.¹⁵ It has also been established that all B cells comprise approximately 10% of circulating PBMCs.¹⁶ Thus, the number of VACV-specific IgG⁺ circulating memory B cells is on the order of 0.01% of total PBMCs. Assuming 1×10⁶ PBMCs per mL of whole blood, the total number of VACV-specific IgG⁺ circulating memory B cells per mL of whole blood is approximately 100. Of that total number of cells, if we assume that VACV L1-specific memory B cells make up 1-10%, then we may expect approximately 1-10 cells per mL of whole blood. Therefore, 100 mL of the ACAM2000 vaccinated donor blood may be processed to isolate 100-1000 VACV L1-specific memory B cells.

### EXAMPLES

### Example 1

Recent efforts have illustrated the unique properties of chitosan, a linear b-1.4-linked polysaccharide generated by the partial deacetylation of chitin, and its use in functional biofabrication.¹⁹⁻²³ Two key properties of this biopolymer include its pH-dependent solubility and the reactivity of its amine groups in enzymatic conjugation. The former property permits the spatially directed assembly of chitosan via electrodepositon by exploiting the pH gradient localized at the cathode surface. Studies have demonstrated the assembly of functionally conjugated chitosan in stable thin films onto micropatterned gold electrodes in a microfluidic device.²⁴ The latter property permits the enzymatic assembly of functionally conjugated chitosan. In 2008, Wu, et. al. described the conjugation of pentatyrosine-tagged modified protein G to an electrodeposited chitosan biopolymer surface mediated by the enzyme tyrosinase and self-assembly of a fluorescently labeled IgG antibody to that functional surface.²⁵ Further demonstrations of the utility of the biopolymer chitosan include the programmable assembly of a functional metabolic pathway enzyme in a pre-packaged, microfluidic reusable bioMEMS device²⁶ and in situ activation and assembly of green fluorescent protein (GFP) onto micropatterned gold electrodes within a multichannel microfluidic device.²⁷ Although chitosan is a preferred polymer, other suitable polymers may be used, for example, polyethylene glycol (PEG), polyacrylamide, etc.

The recruitment of leukocytes such as neutrophils to sites of inflammation is mediated by receptor-ligand interactions. The homing of circulating neutrophils is initiated by the physiological process known as cell rolling. Glycoprotein receptors such as selectins allow for transient adhesive bonds between the leukocyte and the activated endothelium of the blood vessel.²⁸ These bonds have high dissociation rates and are responsive to shear stress.²⁹⁻³⁰ This cell rolling phenomenon has been exploited in vitro to permit the separation and two-dimensional nanomechanical control of cells.³¹⁻³² Fractionation of a mixture of cell lines mediated by immobilized E-selectin in a microfluidic device was demonstrated by Chang, et. al.³³ Separation of stem and progenitor cell populations from other adult bone marrow cells has been accomplished by differential rolling adhesion on L-selectin substrates.³⁴ Studies using IgG coated microspheres in a parallel plate flow chamber were used to correlate increasing critical shear rates for microsphere detachment with increasing ligand surface concentration.³⁵

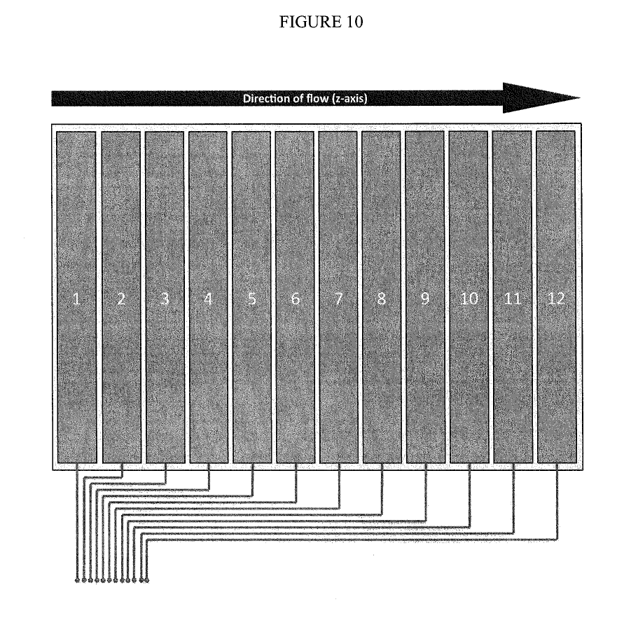

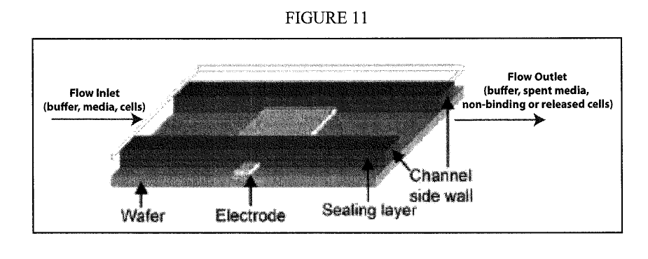

The present invention includes a microfluidic device that will differentially separate antigen-specific B cells based on BCR affinity. As shown in FIG. 10, a microfluidic device of the invention may comprise a plurality of zones and each zone may consist of a rectangular gold electrode or electrode array to permit site specific addressing of functionally-conjugated chitosan mixtures. The flow chamber of the device is designed and will be fabricated to include a sealed flow path to permit sterile operation. The electrodes may be integrated into the flow chamber to allow for electrodepostion of functionally conjugated chitosan or other polymer. Flow chamber design is detailed in FIG. 11.

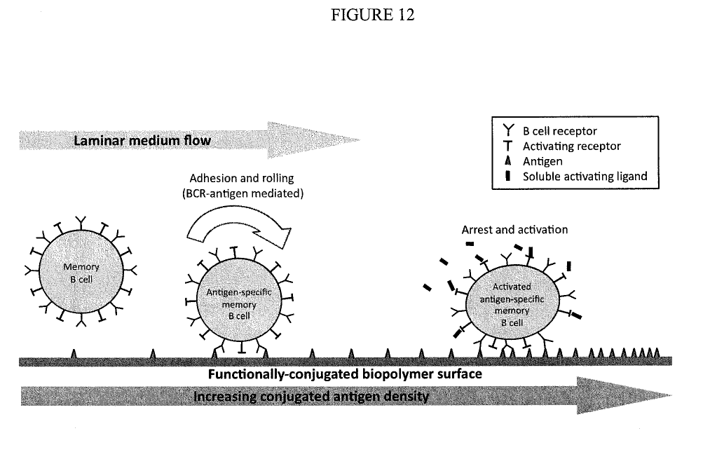

The His-tagged soluble vaccinia virus antigen L1 described above was modified with the addition of a pentatyrosine tag or pentaglutamine to the C-terminus. This antigen may be conjugated to chitosan or other polymer directly or via a tether as described herein. A microfluidic device may be constructed to permit the flow of solutions, culture medium, and B cells across a functional surface. This surface may be electrodeposited with a gradient of VACV L1 antigen or other antigen conjugated chitosan or other polynmer. In some embodiments, the antigen conjugated chitosan or other polymer may be electrodeposited in a step gradient along the z-axis of the device in a thin film. The functional surface is designed to accommodate a spatially distributed set of 100-1000 antigen-specific memory B cells. The cells may be introduced to the device and biopolymer surface by laminar flow of cell culture medium. Memory B cells that display BCRs specific for epitopes on the chitosan-anchored L1 interact with the antigen and roll along the surface in the fashion of leukocyte rolling. Since the surface concentration of L1 antigen along the z-axis can be a gradient, the interactions are additive, cell rolling slows, and a set of antigen-specific B cells will be arrested on the functionalized surface (see FIG. 12). As an additional benefit, the affinity of the BCRs and the resulting secreted mAb can be screened by this differential adhesion mediated separation. Determination of antibody affinity as a function of migration distance can be assayed by surface plasmon resonance measurements (i.e., Biacore analysis) of secreted mAb. An infusion/withdrawal syringe pump (Kent Scientific Corp.) may be used to control flow rate of medium into the flow chamber thereby accurately controlling shear stress across the antigen-conjugated chitosan surface. To prepare concentration surface gradients of conjugated chitosan or other polymer, two or more syringe pumps can be connected to a common inlet tube to the flow chamber. In some embodiments, the electrodeposition surface may be composed of multiple separate electrodes stacked across the flow surface, from the entrance to the exit (see FIG. 10). The number of electrodes will dictate the continuity of the gradient (e.g., the more electrodes, the more continuous the gradient). The syringe pumps control the concentrations of the antigen at each zone. This process can be automated. Initial studies may be performed using microspheres with different anti-L1 mAbs conjugated to the surfaces.

Confirmation of gradient electrodeposition can be accomplished by flowing labeled anti-L1 antibodies over the L1 conjugated biopolymer surface. Initial success criterion is the demonstration of differential migration of a mixture of anti-L1 IgG conjugated microspheres. This indicates that fluid flow induced shear forces allow BCR-antigen bonds to associate and dissociate and cause B cell rolling along the antigen-conjugated biopolymer surface. These microsphere experiments set the initial shear stress range and L1 concentration gradients for immune cell isolation. If the microspheres do not migrate, the addition of a competing antigen-specific mAb or Fab to the mobile phase may cause rolling to occur. Another alternative approach is to begin with static capture of the microspheres to the antigen conjugated surface. Then, begin a gradient of competing antigen-specific mAb or Fab in the mobile phase to induce the detachment of mAb conjugated microspheres with higher off rates. At the end of the gradient, only the microspheres with the lowest off rates and highest affinities will remain. The alternative approaches for the microsphere experiments are translated to immune cell (e.g., B cell) experiments using polyclonal competing antigen-specific mAb or Fab. Yet another alternative approach is to conjugate a selectin to chitosan, add it to the gradient, and reduce the surface concentration of L1 antigen or put the antigen downstream to set up a “capture zone.” This begins the rolling process non-specifically but arrests the antigen-specific cells.³⁶

### Example 2

Cells arrested on the antigen-conjugated chitosan or other polymer surface may be activated to induce differentiation to antibody secreting cells (ASCs) and clonal expansion. Recent and historic studies have identified soluble immune cell (e.g., B cell) activators.³⁷⁻⁴⁵ B cell-activating factor of the TNF family (BAFF) that cooperates with signals emanating from BCR have been shown to stimulate B cell survival, class switch DNA recombination, and antibody production.⁴⁶ In cooperation with IL-10, microbial derived products including CpG-containing oligodeoxynucleotides (ODNs) and bacterial DNA can activate human B cells by turning on the TLR9 pathway.⁴⁷⁻⁴⁹ Recombinant CD40L, IL-4, IL-5, IL-6, IL-15, IL-21 or other supplements may be added to increase activation, differentiation, proliferation, and selection of mAb isotype (i.e., IgG). Once activation and expansion is underway, B cells will form colonies of ASCs. Other suitable activators known to those skilled in the art may be used to activate immune cells of other lineages than B cell.

Prior to experiments in the flow chamber, activation medium formulations may be tested on magnetic bead separated antigen-specific memory B cells. Isolation may be performed and cells seeded in 96-well cell culture plates. A commercially available human immune cell medium such as Lymphocyte Growth Medium-3 (Lonza) may be used to culture the B cells. Matrix experiments can be performed with combinations of the activation supplements cited above and include the addition of the soluble recombinant antigen L1. Initial success criterion includes the activation and expansion of memory B cells as indicated by IgG secretion and cell colony formation, respectively. Once achieved, the activation process may be translated to the flow chamber. Cell culturing considerations such as oxygen mass transport may dictate the modification of the flow chamber to permit gas exchange during B cell activation and expansion.

### Example 3

The flow chamber used to separate, activate, and colonize B cells is designed to permit visualization of colonies microscopically and facilitate colony transfer using a micromanipulator in an aseptic environment. These colonies may be transferred to 96-well plates for additional expansion and screening of supernatants for antigen-specific mAbs via ELISA methods. mAbs in these supernatants can be further characterized for antigen binding/affinity via surface plasmon resonance (Biacore) to determine correlation of antibody affinity as a function of B cell migration distance.

Flow chamber design may be functionally tested for sterility, colony visualization, and transfer. These tests may be performed with antibiotic-free medium and sterile hold steps prior to cell culture experiments. If activation and cultivation of certain antigen-specific B cells in the microfluidic device is not possible, detachment and transfer of cells to plates after capture may be necessary. This alternative approach will dictate additional limiting dilution cloning steps to isolate individual mAbs.

### Example 4

Recombinant soluble VACV L1 was biotinylated using the EZ-Link Micro NHS-PEO₄-Biotinylation Kit (Pierce). Biotinylated L1 was analyzed for reactivity to L1-specific IgG (mAb 7D11 and mAb 10F5) by ELISA. Compared with non-biotinylated L1, the biotinylated reagent exhibited an average 34% and 28% reduction in OD signal for mAb 7D11 and mAb 10F5, respectively. The biotinylated L1 reagent was then analyzed to determine the average number of biotin molecules conjugated to each L1 molecule. This was performed with the Pierce Biotin Quantitation Kit (Cat. No. 28005) which uses HABA (4′-hydroxyazobenzene-2-carboxilic acid) and avidin in a competitive reaction. The result of the experiment showed that an average of 2.2 molecules of biotin are conjugated to each L1 molecule.

In a further characterization experiment, simultaneous reactivity to both anti-L1 and anti-biotin antibodies was demonstrated. Briefly, an L1 capture ELISA was designed by coating a 96-well plate with anti-L1 mAb (7D11). After incubating overnight, the plate was washed and blocked. Blocking buffer was removed and serially diluted samples of biotinylated and non-biotinylated L1 were added to wells. A negative control of blocking buffer with no protein was used to determine background signal. The plate was incubated and then washed. A secondary reagent of anti-biotin mAb was added to all wells. This reagent was sourced from Miltenyi Biotec (Cat. No. 130-090-857). After incubation and washing, rabbit anti-mouse IgG1 was added. After incubation and washing, HRP-conjugated goat anti-rabbit IgG reagents were added. After a final incubation and washing, the plate was developed with an ABTS substrate solution and read on a plate reader at 405 nm. Signals (optical density, OD) from the biotinylated L1 wells were approximately 10 fold higher than both the non-biotinylated wells and the negative (background) wells. Signals from non-biotinylated and negative wells were comparable. These results indicate that the biotinylated L1 reagent can simultaneously bind both an anti-L1 mAb and an anti-biotin mAb and should be suitable for B cell capture experiments.

### Example 5

A healthy human volunteer (Donor BB) was bled for serum and circulating lymphocyte isolation prior to ACAM 2000 smallpox vaccination on Jul. 7, 2009. Approximately 20 mL of blood was drawn into Vacutainer clot activator collection tubes (BD, Ref. 3678230). The contents were centrifuged at 1000×g for 20 minutes. Serum fraction was aspirated, aliquoted at 500 μL into cryotubes, and stored in a liquid nitrogen vapor phase dewar. Approximately 75 mL of blood was drawn into Vacutainer collection tubes with sodium heparin (BD, Ref. 367874). Fifteen (15) mL of Histopaque-1077 was added to each of 5 50-mL conical tubes. Fifteen (15) mL of whole blood was carefully overlaid onto each of the Histopaque-1077 aliquots. The tubes were centrifuged at 400×g for 30 minutes and the peripheral blood mononuclear cell (PBMC) layers were aspirated. The PBMCs were mixed with PBS at a PBS:PBMC ratio of 5:1. The solutions were centrifuged, supernatant was decanted, and PBMCs were resuspended in a total volume of 10 mL of freezing medium (90% FBS/10% DMSO). Cells were aliquoted at 500 μL into cryotubes and stored in a liquid nitrogen vapor phase dewar. Cell count of 1.136×10⁷ viable cells per mL in a total volume of ˜11.5 mL indicated a PBMC recovery of approximately 20% based on a recent CBC analysis of Donor BB.

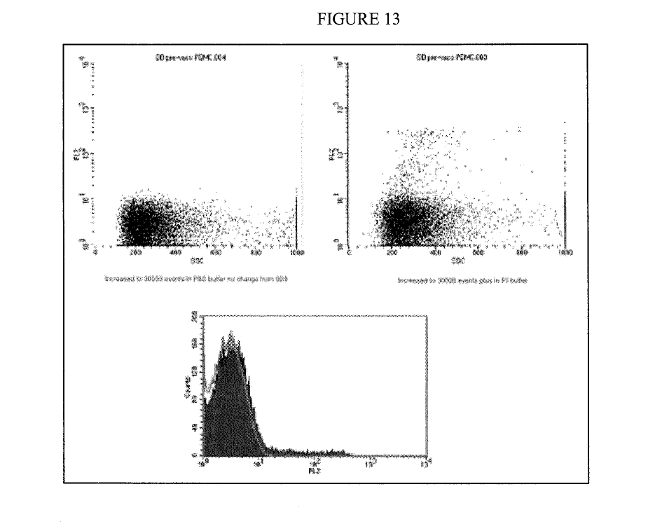

To determine initial suitability and viability of PBMCs for flow cytometer analyses, one aliquot of PBMCs were rapidly thawed and cells were diluted 1:10 into either PBS or PBS with 0.5 μg/mL propidium iodide (PI). These samples were run on a BD FACSVantage instrument. As shown in FIG. 13, cells stained with PI showed nearly 100% viability. PBMCs will be isolated as above at the 21-day post-vaccination time point from Donor BB. In addition, B cell-specific fluorescently labeled Abs will be used to analyze and enrich specific B cell compartments.

### Example 6

Several variants of the VACV L1R antigen gene were designed and generated. Specifically, glutamine- and tyrosine-tagged versions of the L1R ectodomain were engineered and PCR assembled. Each of these variants includes a 3′ (C-terminal) 6-histidine tag for ease of purification. The Tyr and Gln tags are used in studies to conjugate the proteins to chitosan directly and indirectly via a peptide tether, respectively. Chitosan or other biopolymer will be thin-layer electrodeposited onto a microfluidics chip. Once the VACV L1 antigen is conjugated to the chitosan or polymer surface, capture of antigen-specific B cells will be investigated with both model cell lines and human donor circulating lymphocyte pools. Genes for each of these L1 variants were assembled and cloned into a mammalian expression plasmid. Small-scale transient transfection and affinity purification were used to generate quantities of each protein for analysis by ELISA and conjugation studies.

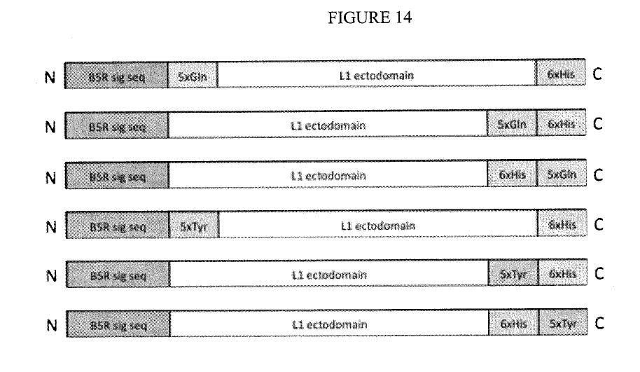

Primers were designed and synthesized and used to generate six PCR products with the various terminal tags (see FIG. 14). This PCR assembly was performed in two steps. First, the L1 ectodomain was PCR amplified from a previous construct pcdna3.1+zeo:intA:VACVsL1R+His#1. Second, the gel purified PCR product was used as a master template with the primer sets in Table 5 to generate all six L1 variants.

TABLE 5
Primers used for PCR assembly of six terminal tagged VACV L1 variants.

| | Sequence, 5′ to 3′ |
|---|---|
| L1 ectodomain | |
| L1-F1 | GGTGCCGCGGCAAGCATACAG |
| L1-R1rev2 | CTGAACTCCTGTACCAGCAACTTGTTTAGGTGC |
| L1 with 5′ 5×Gln tag | |
| and 3′ 6×His tag | |
| L1GlnTag-F2 | CATGAAGCTTCCACCATGAAAACGATTTCCGTTGTT |
| | ACGTTGTTATGCGTACTACCTGCTGTTGTTTATTCAA |
| | CATGTCAGCAGCAGCAGCAGGGTGCCGCGGCAAGCA |
| | TACAG (HindIII) |
| L1HisTag-R2 | CATGGCGGCCGCTCAGTCA [symbol] |
| | [symbol] CTGAACTCCTGTACCAGCAAC (NotI) |
| L1 with 3′ 5×Gln tag | |
| upstream of 3′ 6×His tag | |
| L1-F3 | CATGAAGCTTCCACCATGAAAACGATTTCCGTTGTT |
| | ACGTTGTTATGCGTACTACCTGCTGTTGTTTATTCAA |
| | CATGTGGTGCCGCGGCAAGCATACAG (HindIII) |
| L1GlnTagHisTag-R3 | CATGGCGGCCGCTCAGTCA [symbol] |
| | [symbol] CTGCTGCTGCTGCTGCTGAACTCCTGTACCAGCAAC |
| | (NotI) |
| L1 with 3′ 5×Gln tag | |
| downstream of 3′ 6×His tag | |
| L1-F3 | As above |
| L1HisTagGlnTag-R4 | CATGGCGGCCGCTCAGTCACTGCTGCTGCTGCTG [symbol] |
| | [symbol] CTGAACTCCTGTACCAGCAAC |
| | (NotI) |
| L1 with 5′ 5×Tyr tag | |
| and 3′ 6×His tag | |
| L1TyrTag-F5 | CATGAAGCTTCCACCATGAAAACGATTTCCGTTGTT |
| | ACGTTGTTATGCGTACTACCTGCTGTTGTTTATTCAA |
| | CATGTTACTATTACTATTACGGTGCCGCGGCAAGCATA |
| | CAG (HindIII) |
| L1HisTag-R2 | As above |
| L1 with 3′ 5×Tyr tag | |
| upstream of 3′ 6×His tag | |
| L1-F3 | As above |
| L1TyrTagHisTag-R6 | CATGGCGGCCGCTCAGTCA [symbol] |
| | [symbol] GTAATAGTAATAGTACTGAACTCCTGTACCAGCAAC |
| | (NotI) |
| L1 with 3′ 5×Tyr tag | |
| downstream of 3′ 6×His tag | |
| L1-F3 | As above |
| L1HisTagTyrTag-R7 | CATGGCGGCCGCTCAGTCAGTAATAGTAATAGTA [symbol] |
| | [symbol] CTGAACTCCTGTACCAGCAAC |
| | (NotI) |

Restriction sites are underlined and listed in braces at end of primer. Gln and Tyr tags are italicized. His tag is bold and italicized.

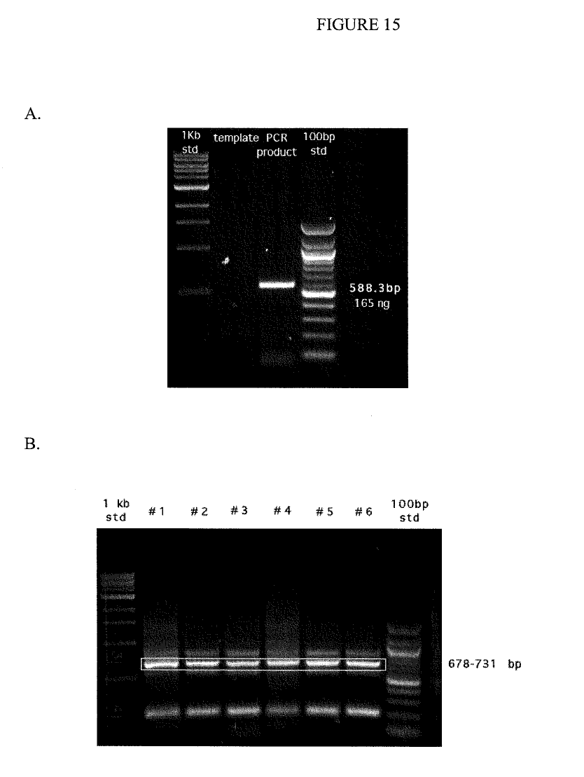

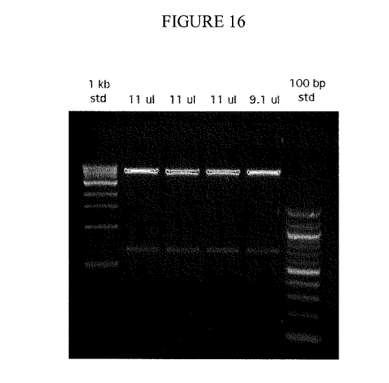

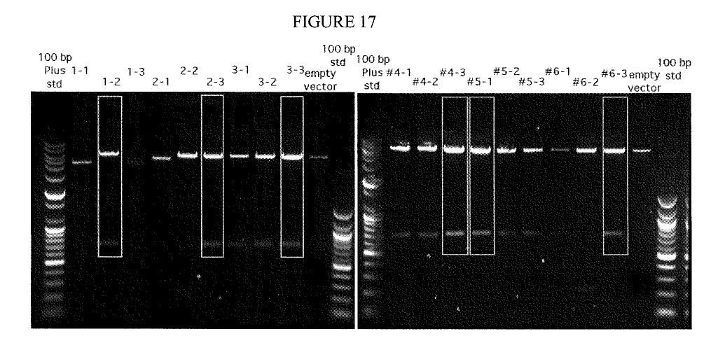

FIG. 15 shows the successful two-step PCR assembly. The PCR product bands indicated by the box in FIG. 15B were excised and gel purified. The original mammalian expression plasmid pcdna3.1+zeo:intA:VACVsL1R+His#1 was restricted with Hind III and Not I and gel purified at large scale to remove the unmodified L1 ectodomain and provide an acceptor vector for each of the six variants (see FIG. 16). The gel purified L1 variants were likewise restricted with Hind III and Not I and then ligated into the acceptor vector. TOP10 competent E. coli (Invitrogen) were transformed with the plasmids and plated on Carb100 LB agar (100 μg/mL carbenicillin). For each of the six variants, 3 colonies were picked and expanded in 3 mL of Carb50 LB broth at 37° C. with shaking for 7 hours. One and one half milliliters of culture were harvested and plasmids were purified using a Qiagen QIAprep Spin Miniprep Kit. One half milliliter of each culture was mixed with an equal volume of 50% glycerol solution and frozen down at −80° C. Plasmid preps were analyzed by restriction endonuclease (REN) using Hind III and Not I. The plasmids indicated by the boxes in FIG. 17 were DNA sequence confirmed using primers that complement regions in the plasmid flanking the insert. All sequences were 100% accurate.

Each of the six constructs was prepped at the midi scale for transient transfection and expression. Briefly, for each construct, 30 mL of Carb50 LB broth was inoculated from a single colony of transformed E. coli. Cultures were incubated overnight at 37° C. with shaking. The next day, cultures were harvested and plasmids were purified using a Qiagen Plasmid Midi Kit. HEK 293T/17 cells were expanded in T-75 flasks in the following growth medium: DMEM-High Glucose (Invitrogen, Cat. No. 11995-040), 10% FBS-Certified (INVG, Cat. No. 16000-069), 2 mM GlutaMAX (INVG, Cat. No. 35050-061), and 1×NEAA (INVG, Cat. No. 11140-050). At passage 11, cells were counted and seeded in 10 cm cell culture dishes at 3×10⁶ viable cells in 15 mL of growth medium. Cells were incubated for two days and were ˜90% confluent. On the day of transfection, medium was replaced with 15 mL of fresh growth medium. For each transfection, 24 μg of plasmid DNA was diluted in 1.5 mL of OptiMEM-I SFM medium (INVG, Cat. No. 31985-062) and 60 μL of Lipofectamine 2000 (INVG, Cat. No. 11668-027) was diluted in 1.5 mL of OptiMEM-I. Solutions were gently combined and incubated at room temperature for 20 minutes. Reactions were added dropwise to each 10 cm cell culture dish and transfections were allowed to proceed for 5.5 days. Supernatants were aspirated and clarified by centrifugation and 0.22 μm filtration.

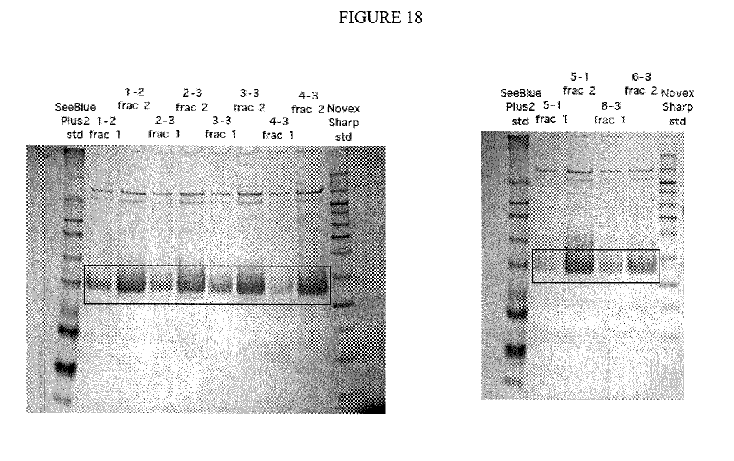

Transiently expressed L1 variants were purified using a HisPur Purification Kit with 3 mL resin bed volume with a spin purification protocol (Pierce, Cat. No. 90092). His-tagged proteins were eluted in two 3 mL fractions using the included elution buffer (50 mM sodium phosphate, 300 mM sodium chloride, 150 mM imidazole; pH 7.4). A total of 26 μL of each fraction was loaded on SDS-PAGE gels in reducing sample buffer (NuPAGE Novex 10% Bis-Tris Gel 1.0 mm, 10 well, INVG, Cat. No. NP0301). Gels were run at 200 V constant for 45 min and stained with coomassie (SimplyBlue SafeStain, INVG, Cat. No. LC6065). Stained SDS-PAGE gels are shown in FIG. 18. Major bands indicated in boxes were 25,881-27,234 Da (mean=26, 601 Da) with a purity of 62-83% by densitometry analysis. Fractions 1 and 2 for each protein were pooled, quantitated by A280, sterile filtered at 0.22 μm, and stored at −20° C.

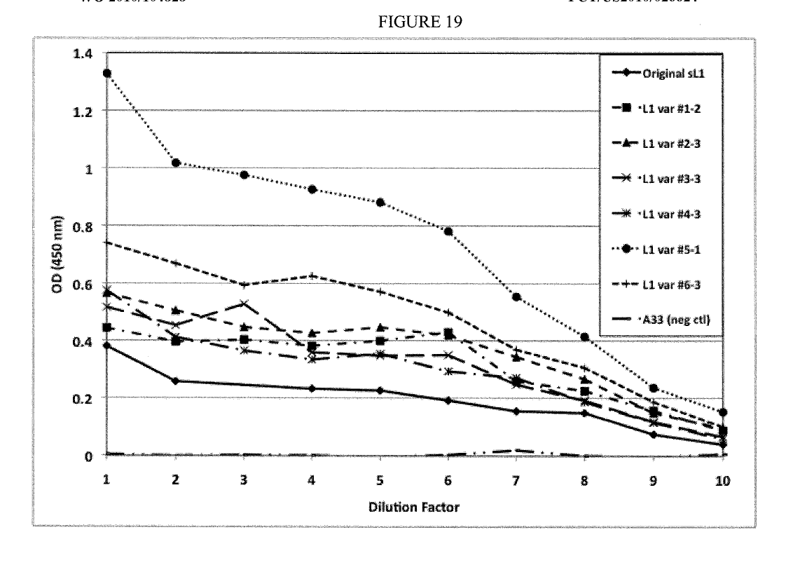

Proteins were analyzed for antibody binding to mAb c7D11 (α-VACV L1 chimeric monoclonal) by ELISA. This analysis will determine if the conformational epitope is conserved indicating proper protein folding (see Su et. al., Virology, 2007, 368 (2):331-41 for Structural basis for the binding of the neutralizing antibody, 7D11, to the poxvirus L1 protein). Immediately prior to freeze-down, pooled final purified proteins were used to coat a 96-well ELISA plate at 100 ng per well in PBS. Each protein was coated in the 12 wells of rows A-F. As a positive control, original soluble L1 (His-tagged only) was coated in the 12 wells of row G at 100 ng per well in PBS. As a negative control, soluble VACV A33 subunit protein was coated in the 12 wells of row H at 100 ng per well in PBS. Plates were incubated overnight at 4° C. The plate was washed and blocked with 5% non-fat dry milk (NFDM), 0.1% TWEEN 20, and 0.01% thymerisol in PBS for 45 minutes with shaking at room temperature. Purified c7D11 mAb was diluted in a 1:2 series in blocking buffer beginning with a concentration of 3000 ng/mL for ten dilutions. Blocking buffer was discarded from the ELISA plate and the c7D11 standard curve dilution series was added to each of the rows (100 μL per well) of the plate reserving the last two wells of each row for blocking buffer only (buffer negative control). The plate was incubated for 45 minutes with shaking at room temperature. Samples were discarded and ELISA plate was washed. Secondary antibody (Anti-Human IgG [Fab Specific]-Peroxidase, antibody produced in goat, Sigma, Cat. No. A0293-1ML) was diluted 1:20,000 in blocking buffer and added to each well (100 μL). The plate was incubated for 45 minutes with shaking at room temperature. Secondary was discarded and ELISA plate was washed. Plate was developed with TMB Liquid Substrate System (Sigma, Cat. No. T0440-100ML, 100 μL per well). Reaction was stopped with the addition of 100 μL per well of 0.5 M H2SO4. ELISA plate was read at 450 nm. FIG. 19 shows the signal curves produced by each row of coated protein (L1 variants, sL1 original, and VACV A33). As seen, all L1 variants demonstrate similar signal response with the mAb c7D11 dilutions and are comparable to or exceed the signal intensities of the original His-tag only sL1. No signal was observed in the A33 or buffer negative control wells. This indicates the preservation of the conformational epitope recognized by mAb c7D11.

### Example 7

Individual L1 variants may be selected based on functional and design criteria, conjugated to chitosan or other polymer, and analyzed for functionality on a chip. Chitosan solutions (1% w/v) may be prepared by dissolving chitosan flakes in an aqueous solution of HCl (1% v/v) and adjusting the pH to 5.6 using 1 M NaOH. A gelatin stock solution (5%) may be prepared by dissolving gelatin in 20 mM phosphate buffered saline (PBS, pH 6.5). Both solutions may be autoclaved at 121° C. for 15 min. Protein assembly may be performed using a chip prepared by patterning gold electrodes onto a silicon wafer using standard microfabrication. To electrodeposit chitosan, the chip may be partially immersed in the chitosan solution (5 mL, 1% w/v, pH 5.6) and an electrode biased to serve as the cathode at a controlled current density of 5 A/m2 for 1 min (typical voltage was less than 2.5 V). After deposition, the electrode may be disconnected from the power supply and the chip washed with deionized water and soaked in PBS (pH 7.4) for 30 min.

To graft the gelatin tethers onto the electrodeposited chitosan films, the chip may be incubated in 3 mL of 20 mM phosphate buffer (pH 6.5) containing tyrosinase (50 U/mL) and gelatin (0.5-4%). After grafting, the chip may be washed with 10 mL of 20 mM phosphate buffer (pH 6.5) containing 0.1% Triton X-100 for three times. Glutamine-tagged L1 variants may be assembled to the tethers by incubating the chip in 3 mL of 20 mM phosphate buffer (pH 6.5) containing microbial transglutaminase (mTG; 1 U/mL) and the gln-tagged L1 (1.3 μM). Reactions may be performed overnight at room temperature.

To demonstrate specific binding of an anti-L1 monoclonal antibody (mAb), the chip may be immersed in a solution containing mAb c7D11 (α-VACV L1 chimeric monoclonal). The chips may be washed and immersed in an anti-human IgG (Fc-specific) fluorescently labeled secondary antibody. After washing, the fluorescence of the electrode addresses may be observed using a fluorescence microscope (Leica; MZFL III). Fluorescence photomicrographs may be obtained for the chip using a digital camera (Spot 32, Diagnostic Instruments) connected to the fluorescence microscope. The fluorescence profiles of the micrographs may be analyzed using Image J software

### Example 8

Capture and expansion of antigen-specific T cells may be accomplished as above with the following modifications: 1) The antigen conjugated to the functional surface in the device may be a peptide with T cell epitopes; 2) The human PBLs may be enriched for T cells (i.e., magnetic bead or flow cytometry methods) prior to introduction into the device for separation; and 3) After T cell separation and arrest on the functionalized surface in the flow chamber, T cells may be activated by the addition of T cell activation medium containing known T cell activation factors. After colonies of clonal T cells form, they may be removed by a micromanipulator, deposited in 96-well cell culture plates, and further expanded.

### REFERENCES

1. Schmaljohn C, Cui Y, Kerby S, Pennock D., and Spik K. Production and characterization of human monoclonal antibody Fab fragments to vaccinia virus from a phage-display combinatorial library. Virology. 1999; 258:189-200.
2. Barbas C F, Kang A S, Lerner R A, and Benkovic S J. Assembly of combinatorial antibody libraries on phage surfaces: The gene III site. PNAS. 1991; 88:7978-82.
3. Guttieri M C, Sinha T, Bookwalter C, Liang M, and Schmaljohn C S. Cassette vectors for conversion of Fab fragments into full-length human IgG1 monoclonal antibodies by expression in stably transformed insect cells. Hybrid Hybridomics. 2003; 22:135-45.
4. Franklin A and Blanden R V. Potential inhibition of somatic hypermutation by nucleoside analogues. Mol. Immun. 2007; 44:666-9.
5. Guay H M, Panarey L, Reed A J, and Caton A J. Specificity-based negative selection of autoreactive B cells during memory formation. J. Immunol. 2004; 173:5485-94.
6. Low N M, Holliger P, and Winter G. Mimicking somatic hypermutation: Affinity maturation of antibodies displayed on bateriophage using a bacterial mutator strain. J. Mol. Biol. 1996; 260:359-68.
7. Steele E J. Reflections on the state of play in somatic hypermutation. Mol. Immunol. 2008; 45:2723-6.
8. Steidl A, Ratsch O, Brocks B, Dürr M, and Thomassen-Wolf E. In vitro affinity maturation of human GM-CSF antibodies by targeted CDR-diversification. Mol. Immun. 2008; 46:135-44.
9. Thompson J, Pope T, Tung J-S, Chan C, Hollis G, Mark G, and Johnson K S. Affinity maturation of a high-affinity human monoclonal antibody against the third hypervariable loop of human immuno deficiency virus: Use of phage display to improve affinity and broaden strain reactivity. J. Mol. Biol. 1996; 256:77-88.
10. Kvell K, Nguyen T H, Salmon P, Glauser F, Werner-Favre C, Barnet M, Schneider P, Trono D, and Zubler R H. Transduction of CpG DNA-stimulated primary human B cells with bicistronic lentivectors. Mol. Immun. 2008; 46:135-44.
11. Lanzavecchia A, Bernasconi N, Traggiai E, Ruprecht C R, Corti D, and Sallusto F. Understanding and making use of human memory B cells. Immunol. Rev. 2006; 211:303-9.
12. Li J, Sai T, Berger M, Chao Q, Davidson D, Deshmukh G, Drozdowski B, Ebel W, Harley S, Henry M, Jacob S, Kline B, Lazo E, Rotella F, Routhier E, Rudolph K, Sage J, Simon P, Yao J, Zhou Y, Kavuru M, Bonfield T, Thormassen M J, Sass P M, Nicolaides N C, and Grasso L. Human antibodies for immunotherapy development generated via a human B cell hybridoma technology. PNAS. 2006; 103:3557-62.
13. Weisner M, Zentz C, Mayr C, Wimmer R, Hammerschmidt W, Zeidler R, and Moosmann A. Conditional immortalization of human B cells by CD40 ligation. PLoS ONE 2008; 1:1-13.
14. Crotty S, Felgner P, Davies H, Glidewell J, Villarreal L, and Ahmed R. Cutting edge: Long-term B cell memory in humans after smallpox vaccination. J. Imm. 2003; 171:4969-73.
15. Klein U, Rajewsky K, and Kuppers R. Human immunoglobulin (Ig)M+IgD+peripheral blood B cells expressing the CD27 cell surface antigen carry somatically mutated variable region genes: CD27 as a general marker for somatically mutated (memory) B cells. J. Exp. Med. 1998; 188:1679-89.
16. Marti G E, Magruder L, Patrick K, Vail M, Schuette W, Keller R, Muirhead K, Horan P, and Gralnick H R. Normal human blood density gradient lymphocyte subset analysis: An Interlaboratory flow cytometric comparison of 85 normal adults. Amer. J. Hema. 1985; 20:41-52.
17. Babcook J S, Leslie K B, Olsen O A, Salmon R A, and Schrader J W. A novel strategy for generating monoclonal antibodies from single, isolated lymphocytes producing antibodies of defined specificities. PNAS. 1996; 93:7843-8.
18. Guan Y, Sajadi M M, Kamin-Lewis R, Fouts T R, Dimitrov A, Zhang Z, Redfield R R, DeVico A L, Gallo R C, and Lewis G K. Discordant memory B cell and circulating anti-Env antibody responses in HIV-1 infection. PNAS. 2009; epub ahead of print.
19. Yi H, Wu L-Q, Bentley W E, Ghodssi R, Rubloff G W, Culver J N, and Payne G F. Biofabrication with chitosan. Macromolecules. 2005; 6:2881-94.
20. Payne G F and Raghavan S R. Chitosan: a soft interconnect for hierarchical assembly of nano-scale components. Soft Matter. 2007; 3:521-7.
21. Lewandowski A T, Bentley W E, Yi H, Rubloff G W, Payne G F, and Ghodssi R. Towards area-based in vitro metabolic engineering: Assembly of Pfs enzyme onto patterned microfabricated chips. Biotech. Prog. 2008; 24:1042-51.
22. Shi X-W, Yang X, Gaskell K J, Liu Y, Kobatake E, Bentley W E, and Payne G F. Reagentless protein assembly triggered by localized electrical signals. Adv. Mat. 2009; 21:984-8.
23. Yang X, Shi X-W, Liu Y, Bentley W E, and Payne G F. Orthogonal enzymatic reactions for the assembly of proteins at electrode addresses. Langmuir. 2009; 25:338-44.
24. Park J J, Luo X, Yi H, Valentine T M, Payne G F, Bentley W E, Ghodssi R, and Rubloff G W. Chitosan-mediated in situ biomolecule assembly in completely packaged microfluidic devices. Lab Chip. 2006; 6:1315-21.
25. Wu H-C, Shi X-W, Tsao C-Y, Lewandowski A T, Fernandes R, Hung C-W, DeShong P, Kobatake E, Valdes J J, Payne G F, and Bentley W E. Biofabrication of antibodies and antigens via IgG-binding domain engineered with activatable pentatyrosine pro-tag. Biotech. Bioeng. 2009; epub ahead of print.
26. Luo X, Lewandowski A T, Yi H, Payne G F, Ghodssi R, Bentley W E, and Rubloff G W. Programmable assembly of a metabolic pathway enzyme in a pre-packaged reusable bioMEMS device. Lab Chip. 2008; 8:420-30.
27. Lewandowski A T, Yi H, Luo X, Payne G F, Ghodssi R, Rubloff G F, and Bentley W E. Protein assembly onto patterned microfabricated devices through enzymatic activation of fusion pro-tag. Biotech. Bioeng. 2008; 99:499-507.
28. Janeway C A, Travers P, Walport M, and Shlomchik M J. Immunobiology: The immune system in health and disease, 6th Ed. New York: Garland Science, 2005.
29. Dong C and Lei X X. Biomechanics of cell rolling: Shear fow, cell-surface adhesion, and cell deformability. J. Biomech. 2000; 33:35-43.
30. Fritz J, Katopodis A G, Kolbinger F, and Anselmetti D. Force-mediated kinetics of single P-selectin/ligand complexes observed by atomic force microscopy. PNAS. 1998; 95:12283-8.
31. Charles N, Liesveld J L, and King M R. Investigating the feasibility of stem cell enrichment mediated by immobilized selectins. Biotech. Prog. 2007; 23:1463-72.
32 Karnik R, Hong S, Zhang H, Mei Y, Anderson D G, Karp J M, and Langer R. Nanomechanical control of cell rolling in two dimensions through surface patterning of receptors. Nano Lett. 2008; 8:1153-8.
33. Chang W C, Lee LP, and Liepmann D. Biomimetic technique for adhesion-based collection and separation of cells in a microfluidic channel. Lab Chip. 2005; 5:64-73.
34. Greenberg A W and Hammer D A. Cell separation mediated by differential rolling adhesion. Biotech. Bioeng. 2001; 73:111-24.
35. Gao B and Jin G. Study of interaction force between antigen and antibody using flow chamber method. Proc. of IEEE EMB 27th Ann. Conf. 2005; 7584-7.
36. Hong S, Lee D, Zhang H, Zhang J Q, Resvick J N, Khademhosseini A, King M R, Langer R, and Karp J M. Covalent immobilization of P-selectin enhances cell rolling. Langmuir. 2007; 23:12261-8.
37. Bagley K C, Abdelwahab S F, Tuskan R G, and Lewis G K. Cholera toxin indirectly activates human monocyte-derived dendritic cells in vitro through the production of soluble factors, including prostaglandin E2 and nitric oxide. Clinical and Vaccine Immunology 2006; 13:106-15.
38. Bagley K C, Abdelwahab S F, Tuskan R G, and Lewis G K. Pasteurella multocida toxin activates human monocyte-derived and murine bone marrow-derived dendritic cells in vitro but suppresses antibody production in vivo. Infection and Immunity 2005; 73:413-21.
39. Bagley K C, Abdelwahab S F, Tuskan R G, and Lewis G K. Calcium signaling through phospholipase C activates dendritic cells to mature and is necessary for the activation and maturation of dendritic cells induced by diverse agonists. Clinical and Diagnostic Laboratory Immunology 2004; 11:77-82.
40. Bagley K C, Abdelwahab S F, Tuskan R G, Fouts T R, and Lewis G K. Pertussis toxin and the adenylate cyclase toxin from Bordetella pertussis activate human monocyte-derived dendritic cells and dominantly inhibit cytokine production through a cAMP-dependent pathway. J. Leukocyte Biol. 2002; 72:962-9.
41. Brunswick M, June C H, Finkelman F D, Dintzis H M, Inman J K and Mond J J. Surface immunoglobulin-mediated B-cell activation in the absence of detectable elevations in intracellular ionized calcium: A model for T-cell independence B-cell activation. PNAS 1989; 86:6724-8.
42. Hebeis B J, Klenovsek K, Rohwer P, Ritter U, Schneider A, Mach M, and Winkler T H. Activation of virus-specific memory B cells in the absence of T cell help. J. Exp. Med. 2004; 199:593-602.
43. Klaus S J, Pinchuk L M, Ochs H D, Law C-L, Fanslow W C, Armitage R J, and Clark E A. Costimulation through CD28 enhances T cell-dependent B cell activation via CD40-CD40L interaction. J. Immun. 1994; 152:5643-52.
44. Li W. Synergistic antibody induction by antigen-CD40 ligand fusion protein as improved immunogen. Immun. 2005; 115:215-22.
45. Li X, Vanitha D J, Joo H M, He Y, Rouse B T, and Sangster M Y. A strategy for selective, CD4+ T cell-independent activation of virus-specific memory B cells for limiting dilution analysis. J. Immun. Meth. 2006; 313:110-8.
46. Schneider P, MacKay F, Steiner V, Hofmann K, Bodmer J L, Holler N, Ambrose C, Lawton P, Bixler S, and Acha-Orbea S. BAFF, a novel ligand of the tumor necrosis factor family, stimulates B cell growth. J. Exp. Med. 1999. 189:1747.
47. He B, Qiao X, and Cerutti A. CpG DNA induces IgG class switch DNA recombination by activating human B cells through an innate pathway that requires TLR9 and cooperates with IL-10. J. Immun. 2004; 173:4479-91.
48. Huggins J, Pellegrin T, Felgar R E, Wei C, Brown M, Zheng B, Milner E C B, Bernstein S H, Sanz I, and Zand M S. CpG DNA activation and plasma-cell differentiation of CD27-naïve humand B cells. Blood. 2007; 109:1611-9
49. Richard K, Pierce S K, and Song W. The agonists of TLR4 and 9 are sufficient to activate memory B cells to differentiate into plasma cells in vitro but not in vivo. J. Immunol. 2008; 181:1746-52.

While the foregoing invention has been described in some detail for purposes of clarity and understanding, it will be appreciated by one skilled in the art from a reading of this disclosure that various changes in form and detail can be made without departing from the true scope of the invention and appended claims. All patents and publications cited herein are entirely incorporated herein by reference.

What is claimed is:

1. A device for the separation of immune cells by interactions of receptors of said immune cells with ligands wherein the ligands are immobilized on a functionalized surface of the device and the device is adapted to flow the immune cells across the functionalized surface.

2. The device of claim 1, wherein said device is a flow cell or microfluidics device.

3. The device of claim 1, wherein said immune cells are B cells.

4. The device of claim 1, wherein said immune cell receptors are B cell receptors.

5. The device of claim 1, wherein said ligands are antigens with B cell epitopes.

6. The device of claim 1, wherein said immune cells are T cells.

7. The device of claim 1, wherein said immune cell receptors are T cell receptors.

8. The device of claim 1, wherein said ligands are antigens with T cell epitopes.

9. The device of claim 1, wherein said functionalized surface is a polymer conjugated to said ligands.

10. A method for isolating immune cells, comprising:
contacting a solution comprising the immune cells with a device according to claim 1.

11. A method according to claim 10, wherein the immune cells are arrested on said functionalized surface and are caused to proliferate by induction of activation.

12. A method according to claim 11, wherein viable activated proliferating immune cell colonies are recovered from the device.

13. A method according to claim 12, wherein colonies are recovered by releasing the ligand from the polymer.

14. A method according to claim 12, wherein the colonies are mechanically removed.
